SEQUENCE LISTING (112 sequences)

SEQ ID NO: 1 - DNA, 33 bp, Artificial Sequence
  Synthetic Nucleic Acid
agatcggcagcgtcagatgtgtataagagacag

SEQ ID NO: 2 - DNA, 55 bp, Artificial Sequence
  Synthetic Nucleic Acid
agatcggcagcgtcagatgtgtataagagacaggagtagttgggattataggtgt

SEQ ID NO: 3 - DNA, 57 bp, Artificial Sequence
  Synthetic Nucleic Acid
agatcggcagcgtcagatgtgtataagagacagttagaaatttagttgtagaggggg

SEQ ID NO: 4 - DNA, 56 bp, Artificial Sequence
  Synthetic Nucleic Acid
agatcggcagcgtcagatgtgtataagagacaggaggttagggttttagattggga

SEQ ID NO: 5 - DNA, 55 bp, Artificial Sequence
  Synthetic Nucleic Acid
agatcggcagcgtcagatgtgtataagagacaggtaaygaattggtagagtttta

SEQ ID NO: 6 - DNA, 57 bp, Artificial Sequence
  Synthetic Nucleic Acid
agatcggcagcgtcagatgtgtataagagacagtgagggtaagaattatttagaggt

SEQ ID NO: 7 - DNA, 58 bp, Artificial Sequence
  Synthetic Nucleic Acid
agatcggcagcgtcagatgtgtataagagacagagggttaaagaagagaatgatttat

SEQ ID NO: 8 - DNA, 61 bp, Artificial Sequence
  Synthetic Nucleic Acid
agatcggcagcgtcagatgtgtataagagacaggagggttgaatattaaaaatagtagggt

SEQ ID NO: 9 - DNA, 62 bp, Artificial Sequence
  Synthetic Nucleic Acid
agatcggcagcgtcagatgtgtataagagacagggataattataagaattgtaaaggaggat

SEQ ID NO: 10 - DNA, 58 bp, Artificial Sequence
  Synthetic Nucleic Acid
agatcggcagcgtcagatgtgtataagagacagggtagttggaaatggtaaatttgag

SEQ ID NO: 11 - DNA, 58 bp, Artificial Sequence
  Synthetic Nucleic Acid
agatcggcagcgtcagatgtgtataagagacaggagttatgttatgggagtaagtggg

SEQ ID NO: 12 - DNA, 18 bp, Artificial Sequence
  Synthetic Nucleic Acid
agatcgctcttccgatct

SEQ ID NO: 13 - DNA, 45 bp, Artificial Sequence
  Synthetic Nucleic Acid
agatcgctcttccgatctatatatatcaaacactrgacttaaaat

SEQ ID NO: 14 - DNA, 42 bp, Artificial Sequence
  Synthetic Nucleic Acid
agatcgctcttccgatctccttaaaacaaacttatcttctcc

SEQ ID NO: 15 - DNA, 46 bp, Artificial Sequence
  Synthetic Nucleic Acid
agatcgctcttccgatctcaccttaacaaataaaataataattcac

SEQ ID NO: 16 - DNA, 42 bp, Artificial Sequence
  Synthetic Nucleic Acid
agatcgctcttccgatcttatactaactcccttcaaccatta

SEQ ID NO: 17 - DNA, 41 bp, Artificial Sequence
  Synthetic Nucleic Acid
agatcgctcttccgatctctacccacacctaccaaacctaa

SEQ ID NO: 18 - DNA, 44 bp, Artificial Sequence
  Synthetic Nucleic Acid
agatcgctcttccgatctatcaaaaataattctaaaaatataca

SEQ ID NO: 19 - DNA, 48 bp, Artificial Sequence
  Synthetic Nucleic Acid
agatcgctcttccgatctaccaacttctatataactaataaatacaca

SEQ ID NO: 20 - DNA, 43 bp, Artificial Sequence
  Synthetic Nucleic Acid
agatcgctcttccgatctaaaattcacttctaaatttaaacca

SEQ ID NO: 21 - DNA, 48 bp, Artificial Sequence
  Synthetic Nucleic Acid
agatcgctcttccgatctaaaataatcttcatcaaattaataaaaaca

SEQ ID NO: 22 - DNA, 42 bp, Artificial Sequence
  Synthetic Nucleic Acid
agatcgctcttccgatctacaccaaaaacaatttaataaaca

SEQ ID NO: 23 - DNA, 56 bp, Artificial Sequence
  Synthetic Nucleic Acid
  modified_base (30)..(37): Illumina Index label sequence of 8nt
  misc_feature (30)..(37): n is a, c, g, or t
aatgatacggcgaccaccgagatctacacnnnnnnnntcgtcggcagcgtcagatg

SEQ ID NO: 24 - DNA, 23 bp, Artificial Sequence
  Synthetic Nucleic Acid
tacacatctgacgctgccgacga

SEQ ID NO: 25 - DNA, 64 bp, Artificial Sequence
  Synthetic Nucleic Acid
  modified_base (33)..(40): Illumina Index label sequence of 8nt
  misc_feature (33)..(40): n is a, c, g, or t
atcggaagagcacacgtctgaactccagtcacnnnnnnnnatctcgtatgccgtcttctgcttg

SEQ ID NO: 26 - DNA, 29 bp, Artificial Sequence
  Synthetic Nucleic Acid
gtgactggagttcagacgtgtgctcttcc

SEQ ID NO: 27 - DNA, 54 bp, Artificial Sequence
  Synthetic Nucleic Acid
agatcggcagcgtcagatgtgtataagagacagttagggttttagattgggagg

SEQ ID NO: 28 - DNA, 44 bp, Artificial Sequence
  Synthetic Nucleic Acid
agatcgctcttccgatctttttaccaaaactaatactaacaact

SEQ ID NO: 29 - DNA, 58 bp, Artificial Sequence
  Synthetic Nucleic Acid
agatcggcagcgtcagatgtgtataagagacaggttagggaagttgatgttaggaaat

SEQ ID NO: 30 - DNA, 41 bp, Artificial Sequence
  Synthetic Nucleic Acid
agatcgctcttccgatctaatcaatctctctaaaccaaaaa

SEQ ID NO: 31 - DNA, 62 bp, Artificial Sequence
  Synthetic Nucleic Acid
agatcggcagcgtcagatgtgtataagagacagtagttatatggaaagttgagatagaagga

SEQ ID NO: 32 - DNA, 47 bp, Artificial Sequence
  Synthetic Nucleic Acid
agatcgctcttccgatctaatacaaatcaataaatttacatacaaaa

SEQ ID NO: 33 - DNA, 62 bp, Artificial Sequence
  Synthetic Nucleic Acid
agatcggcagcgtcagatgtgtataagagacagaagaataatttaataggattggaaggaat

SEQ ID NO: 34 - DNA, 47 bp, Artificial Sequence
  Synthetic Nucleic Acid
agatcgctcttccgatctacataataaaaccctatctctactaaaaa

SEQ ID NO: 35 - DNA, 56 bp, Artificial Sequence
  Synthetic Nucleic Acid
agatcggcagcgtcagatgtgtataagagacagtataggtgattttaggggtgaga

SEQ ID NO: 36 - DNA, 51 bp, Artificial Sequence
  Synthetic Nucleic Acid
agatcgctcttccgatcttaaatccttaaataaactacataaaaattttcc

SEQ ID NO: 37 - DNA, 62 bp, Artificial Sequence
  Synthetic Nucleic Acid
agatcggcagcgtcagatgtgtataagagacaggaggtagtaatagggaaaatagttattgg

SEQ ID NO: 38 - DNA, 42 bp, Artificial Sequence
  Synthetic Nucleic Acid
agatcgctcttccgatctaccaactatacctctacatcaaaa

SEQ ID NO: 39 - DNA, 57 bp, Artificial Sequence
  Synthetic Nucleic Acid
agatcggcagcgtcagatgtgtataagagacagaagggggaattttagttttaggaa

SEQ ID NO: 40 - DNA, 48 bp, Artificial Sequence
  Synthetic Nucleic Acid
agatcgctcttccgatctaaaacctatatctctaataaaaactcaata

SEQ ID NO: 41 - DNA, 54 bp, Artificial Sequence
  Synthetic Nucleic Acid
agatcggcagcgtcagatgtgtataagagacagtttgttttaggaaagaggtgg

SEQ ID NO: 42 - DNA, 42 bp, Artificial Sequence
  Synthetic Nucleic Acid
agatcgctcttccgatctaaaaccccaacattcaattaaaaa

SEQ ID NO: 43 - DNA, 63 bp, Artificial Sequence
  Synthetic Nucleic Acid
agatcggcagcgtcagatgtgtataagagacagaataatgtaataagaataaaaggtaaggtt

SEQ ID NO: 44 - DNA, 44 bp, Artificial Sequence
  Synthetic Nucleic Acid
agatcgctcttccgatctaacaccatctcaactcactacaaact

SEQ ID NO: 45 - DNA, 53 bp, Artificial Sequence
  Synthetic Nucleic Acid
agatcggcagcgtcagatgtgtataagagacaggagtattggggatttagggg

SEQ ID NO: 46 - DNA, 44 bp, Artificial Sequence
  Synthetic Nucleic Acid
agatcgctcttccgatctccccaacctctaatatatatacccaa

SEQ ID NO: 47 - DNA, 62 bp, Artificial Sequence
  Synthetic Nucleic Acid
agatcggcagcgtcagatgtgtataagagacagggataaagtaaaggagatattgtatggaa

SEQ ID NO: 48 - DNA, 48 bp, Artificial Sequence
  Synthetic Nucleic Acid
agatcgctcttccgatctaaccacaaataaaatataaatactcataaa

SEQ ID NO: 49 - DNA, 59 bp, Artificial Sequence
  Synthetic Nucleic Acid
agatcggcagcgtcagatgtgtataagagacagggaggaaagagaatatttgatatttg

SEQ ID NO: 50 - DNA, 43 bp, Artificial Sequence
  Synthetic Nucleic Acid
agatcgctcttccgatctaacctctttatttacaaacctaaac

SEQ ID NO: 51 - DNA, 59 bp, Artificial Sequence
  Synthetic Nucleic Acid
agatcggcagcgtcagatgtgtataagagacagtattttaatctcctcaccaacaaaaa

SEQ ID NO: 52 - DNA, 43 bp, Artificial Sequence
  Synthetic Nucleic Acid
agatcgctcttccgatctcacttcctaaaacrgaaaaattcta

SEQ ID NO: 53 - DNA, 4 bp, Artificial Sequence
  Synthetic Nucleic Acid
tgta

SEQ ID NO: 54 - DNA, 4 bp, Artificial Sequence
  Synthetic Nucleic Acid
taca

SEQ ID NO: 55 - DNA, 3 bp, Artificial Sequence
  Synthetic Nucleic Acid
gat

SEQ ID NO: 56 - DNA, 3 bp, Artificial Sequence
  Synthetic Nucleic Acid
atc

SEQ ID NO: 57 - DNA, 17 bp, Artificial Sequence
  Synthetic Nucleic Acid
  modified_base (5)..(5): n=dITP
  misc_feature (5)..(5): n is a, c, g, or t
atgtntataagagacag

SEQ ID NO: 58 - DNA, 8 bp, Artificial Sequence
  Synthetic Nucleic Acid
  modified_base (5)..(5): n=dITP
  misc_feature (5)..(5): n is a, c, g, or t
ttccnatc

SEQ ID NO: 59 - DNA, 39 bp, Artificial Sequence
  Synthetic Nucleic Acid
  modified_base (5)..(5): n=dITP
  misc_feature (5)..(5): n is a, c, g, or t
atgtntataagagacaggagtagttgggattataggtgt

SEQ ID NO: 60 - DNA, 35 bp, Artificial Sequence
  Synthetic Nucleic Acid
  modified_base (5)..(5): n=dITP
  misc_feature (5)..(5): n is a, c, g, or t
ttccnatcatatatatcaaacactrgacttaaaat

SEQ ID NO: 61 - DNA, 41 bp, Artificial Sequence
  Synthetic Nucleic Acid
  modified_base (5)..(5): n=dITP
  misc_feature (5)..(5): n is a, c, g, or t
atgtntataagagacagttagaaatttagttgtagaggggg

SEQ ID NO: 62 - DNA, 32 bp, Artificial Sequence
  Synthetic Nucleic Acid
  modified_base (5)..(5): n=dITP
  misc_feature (5)..(5): n is a, c, g, or t
ttccnatcccttaaaacaaacttatcttctcc

SEQ ID NO: 63 - DNA, 40 bp, Artificial Sequence
  Synthetic Nucleic Acid
  modified_base (5)..(5): n=dITP
  misc_feature (5)..(5): n is a, c, g, or t
atgtntataagagacaggaggttagggttttagattggga

SEQ ID NO: 64 - DNA, 36 bp, Artificial Sequence
  Synthetic Nucleic Acid
  modified_base (5)..(5): n=dITP
  misc_feature (5)..(5): n is a, c, g, or t
ttccnatccaccttaacaaataaaataataattcac

SEQ ID NO: 65 - DNA, 39 bp, Artificial Sequence
  Synthetic Nucleic Acid
  modified_base (5)..(5): n=dITP
  misc_feature (5)..(5): n is a, c, g, or t
atgtntataagagacaggtaaygaattggtagagtttta

SEQ ID NO: 66 - DNA, 32 bp, Artificial Sequence
  Synthetic Nucleic Acid
  modified_base (5)..(5): n=dITP
  misc_feature (5)..(5): n is a, c, g, or t
ttccnatctatactaactcccttcaaccatta

SEQ ID NO: 67 - DNA, 41 bp, Artificial Sequence
  Synthetic Nucleic Acid
  modified_base (5)..(5): n=dITP
  misc_feature (5)..(5): n is a, c, g, or t
atgtntataagagacagtgagggtaagaattatttagaggt

SEQ ID NO: 68 - DNA, 31 bp, Artificial Sequence
  Synthetic Nucleic Acid
  modified_base (5)..(5): n=dITP
  misc_feature (5)..(5): n is a, c, g, or t
ttccnatcctacccacacctaccaaacctaa

SEQ ID NO: 69 - DNA, 42 bp, Artificial Sequence
  Synthetic Nucleic Acid
  modified_base (5)..(5): n=dITP
  misc_feature (5)..(5): n is a, c, g, or t
atgtntataagagacagagggttaaagaagagaatgatttat

SEQ ID NO: 70 - DNA, 34 bp, Artificial Sequence
  Synthetic Nucleic Acid
  modified_base (5)..(5): n=dITP
  misc_feature (5)..(5): n is a, c, g, or t
ttccnatcatcaaaaataattctaaaaatataca

SEQ ID NO: 71 - DNA, 45 bp, Artificial Sequence
  Synthetic Nucleic Acid
  modified_base (5)..(5): n=dITP
  misc_feature (5)..(5): n is a, c, g, or t
atgtntataagagacaggagggttgaatattaaaaatagtagggt

SEQ ID NO: 72 - DNA, 38 bp, Artificial Sequence
  Synthetic Nucleic Acid
  modified_base (5)..(5): n=dITP
  misc_feature (5)..(5): n is a, c, g, or t
ttccnatcaccaacttctatataactaataaatacaca

SEQ ID NO: 73 - DNA, 46 bp, Artificial Sequence
  Synthetic Nucleic Acid
  modified_base (5)..(5): n=dITP
  misc_feature (5)..(5): n is a, c, g, or t
atgtntataagagacagggataattataagaattgtaaaggaggat

SEQ ID NO: 74 - DNA, 33 bp, Artificial Sequence
  Synthetic Nucleic Acid
  modified_base (5)..(5): n=dITP
  misc_feature (5)..(5): n is a, c, g, or t
ttccnatcaaaattcacttctaaatttaaacca

SEQ ID NO: 75 - DNA, 42 bp, Artificial Sequence
  Synthetic Nucleic Acid
  modified_base (5)..(5): n=dITP
  misc_feature (5)..(5): n is a, c, g, or t
atgtntataagagacagggtagttggaaatggtaaatttgag

SEQ ID NO: 76 - DNA, 38 bp, Artificial Sequence
  Synthetic Nucleic Acid
  modified_base (5)..(5): n=dITP
  misc_feature (5)..(5): n is a, c, g, or t
ttccnatcaaaataatcttcatcaaattaataaaaaca

SEQ ID NO: 77 - DNA, 42 bp, Artificial Sequence
  Synthetic Nucleic Acid
  modified_base (5)..(5): n=dITP
  misc_feature (5)..(5): n is a, c, g, or t
atgtntataagagacaggagttatgttatgggagtaagtggg

SEQ ID NO: 78 - DNA, 32 bp, Artificial Sequence
  Synthetic Nucleic Acid
  modified_base (5)..(5): n=dITP
  misc_feature (5)..(5): n is a, c, g, or t
ttccnatcacaccaaaaacaatttaataaaca

SEQ ID NO: 79 - DNA, 38 bp, Artificial Sequence
  Synthetic Nucleic Acid
  modified_base (5)..(5): n=dITP
  misc_feature (5)..(5): n is a, c, g, or t
atgtntataagagacagttagggttttagattgggagg

SEQ ID NO: 80 - DNA, 34 bp, Artificial Sequence
  Synthetic Nucleic Acid
  modified_base (5)..(5): n=dITP
  misc_feature (5)..(5): n is a, c, g, or t
ttccnatcttttaccaaaactaatactaacaact

SEQ ID NO: 81 - DNA, 42 bp, Artificial Sequence
  Synthetic Nucleic Acid
  modified_base (5)..(5): n=dITP
  misc_feature (5)..(5): n is a, c, g, or t
atgtntataagagacaggttagggaagttgatgttaggaaat

SEQ ID NO: 82 - DNA, 31 bp, Artificial Sequence
  Synthetic Nucleic Acid
  modified_base (5)..(5): n=dITP
  misc_feature (5)..(5): n is a, c, g, or t
ttccnatcaatcaatctctctaaaccaaaaa

SEQ ID NO: 83 - DNA, 46 bp, Artificial Sequence
  Synthetic Nucleic Acid
  modified_base (5)..(5): n=dITP
  misc_feature (5)..(5): n is a, c, g, or t
atgtntataagagacagtagttatatggaaagttgagatagaagga

SEQ ID NO: 84 - DNA, 37 bp, Artificial Sequence
  Synthetic Nucleic Acid
  modified_base (5)..(5): n=dITP
  misc_feature (5)..(5): n is a, c, g, or t
ttccnatcaatacaaatcaataaatttacatacaaaa

SEQ ID NO: 85 - DNA, 46 bp, Artificial Sequence
  Synthetic Nucleic Acid
  modified_base (5)..(5): n=dITP
  misc_feature (5)..(5): n is a, c, g, or t
atgtntataagagacagaagaataatttaataggattggaaggaat

SEQ ID NO: 86 - DNA, 37 bp, Artificial Sequence
  Synthetic Nucleic Acid
  modified_base (5)..(5): n=dITP
  misc_feature (5)..(5): n is a, c, g, or t
ttccnatcacataataaaaccctatctctactaaaaa

SEQ ID NO: 87 - DNA, 40 bp, Artificial Sequence
  Synthetic Nucleic Acid
  modified_base (5)..(5): n=dITP
  misc_feature (5)..(5): n is a, c, g, or t
atgtntataagagacagtataggtgattttaggggtgaga

SEQ ID NO: 88 - DNA, 45 bp, Artificial Sequence
  Synthetic Nucleic Acid
  modified_base (5)..(5): n=dITP
agatcgctcttccgatcttaaatccttaaataaactacataaaaa

SEQ ID NO: 89 - DNA, 46 bp, Artificial Sequence
  Synthetic Nucleic Acid
  modified_base (5)..(5): n=dITP
  misc_feature (5)..(5): n is a, c, g, or t
atgtntataagagacaggaggtagtaatagggaaaatagttattgg

SEQ ID NO: 90 - DNA, 32 bp, Artificial Sequence
  Synthetic Nucleic Acid
  modified_base (5)..(5): n=dITP
  misc_feature (5)..(5): n is a, c, g, or t
ttccnatcaccaactatacctctacatcaaaa

SEQ ID NO: 91 - DNA, 41 bp, Artificial Sequence
  Synthetic Nucleic Acid
  modified_base (5)..(5): n=dITP
  misc_feature (5)..(5): n is a, c, g, or t
atgtntataagagacagaagggggaattttagttttaggaa

SEQ ID NO: 92 - DNA, 38 bp, Artificial Sequence
  Synthetic Nucleic Acid
  modified_base (5)..(5): n=dITP
  misc_feature (5)..(5): n is a, c, g, or t
ttccnatcaaaacctatatctctaataaaaactcaata

SEQ ID NO: 93 - DNA, 38 bp, Artificial Sequence
  Synthetic Nucleic Acid
  modified_base (5)..(5): n=dITP
  misc_feature (5)..(5): n is a, c, g, or t
atgtntataagagacagtttgttttaggaaagaggtgg

SEQ ID NO: 94 - DNA, 32 bp, Artificial Sequence
  Synthetic Nucleic Acid
  modified_base (5)..(5): n=dITP
  misc_feature (5)..(5): n is a, c, g, or t
ttccnatcaaaaccccaacattcaattaaaaa

SEQ ID NO: 95 - DNA, 47 bp, Artificial Sequence
  Synthetic Nucleic Acid
  modified_base (5)..(5): n=dITP
  misc_feature (5)..(5): n is a, c, g, or t
atgtntataagagacagaataatgtaataagaataaaaggtaaggtt

SEQ ID NO: 96 - DNA, 34 bp, Artificial Sequence
  Synthetic Nucleic Acid
  modified_base (5)..(5): n=dITP
  misc_feature (5)..(5): n is a, c, g, or t
ttccnatcaacaccatctcaactcactacaaact

SEQ ID NO: 97 - DNA, 37 bp, Artificial Sequence
  Synthetic Nucleic Acid
  modified_base (5)..(5): n=dITP
  misc_feature (5)..(5): n is a, c, g, or t
atgtntataagagacaggagtattggggatttagggg

SEQ ID NO: 98 - DNA, 34 bp, Artificial Sequence
  Synthetic Nucleic Acid
  modified_base (5)..(5): n=dITP
  misc_feature (5)..(5): n is a, c, g, or t
ttccnatcccccaacctctaatatatatacccaa

SEQ ID NO: 99 - DNA, 46 bp, Artificial Sequence
  Synthetic Nucleic Acid
  modified_base (5)..(5): n=dITP
  misc_feature (5)..(5): n is a, c, g, or t
atgtntataagagacagggataaagtaaaggagatattgtatggaa

SEQ ID NO: 100 - DNA, 38 bp, Artificial Sequence
  Synthetic Nucleic Acid
  modified_base (5)..(5): n=dITP
  misc_feature (5)..(5): n is a, c, g, or t
ttccnatcaaccacaaataaaatataaatactcataaa

SEQ ID NO: 101 - DNA, 43 bp, Artificial Sequence
  Synthetic Nucleic Acid
  modified_base (5)..(5): n=dITP
  misc_feature (5)..(5): n is a, c, g, or t
atgtntataagagacagggaggaaagagaatatttgatatttg

SEQ ID NO: 102 - DNA, 33 bp, Artificial Sequence
  Synthetic Nucleic Acid
  modified_base (5)..(5): n=dITP
  misc_feature (5)..(5): n is a, c, g, or t
ttccnatcaacctctttatttacaaacctaaac

SEQ ID NO: 103 - DNA, 43 bp, Artificial Sequence
  Synthetic Nucleic Acid
  modified_base (5)..(5): n=dITP
  misc_feature (5)..(5): n is a, c, g, or t
atgtntataagagacagtattttaatctcctcaccaacaaaaa

SEQ ID NO: 104 - DNA, 33 bp, Artificial Sequence
  Synthetic Nucleic Acid
  modified_base (5)..(5): n=dITP
  misc_feature (5)..(5): n is a, c, g, or t
ttccnatccacttcctaaaacrgaaaaattcta

SEQ ID NO: 105 - DNA, 58 bp, Artificial Sequence
  Synthetic Nucleic Acid
  modified_base (30)..(37): Illumina Index label sequence of 8nt
  misc_feature (30)..(37): n is a, c, g, or t
aatgatacggcgaccaccgagatctacacnnnnnnnntcgtcggcagcgtcagatgtg

SEQ ID NO: 106 - DNA, 16 bp, Artificial Sequence
  Synthetic Nucleic Acid
ctgacgctgccgacga

SEQ ID NO: 107 - DNA, 57 bp, Artificial Sequence
  Synthetic Nucleic Acid
  modified_base (26)..(33): Illumina Index label sequence of 8nt
  misc_feature (26)..(33): n is a, c, g, or t
gagcacacgtctgaactccagtcacnnnnnnnnatctcgtatgccgtcttctgcttg

SEQ ID NO: 108 - DNA, 30 bp, Artificial Sequence
  Synthetic Nucleic Acid
gtgactggagttcagacgtgtgctcttccg

SEQ ID NO: 109 - DNA, 5 bp, Artificial Sequence
  Synthetic Nucleic Acid
cacat

SEQ ID NO: 110 - DNA, 5 bp, Artificial Sequence
  Synthetic Nucleic Acid
atgtg

SEQ ID NO: 111 - DNA, 5 bp, Artificial Sequence
  Synthetic Nucleic Acid
cggaa

SEQ ID NO: 112 - DNA, 5 bp, Artificial Sequence
  Synthetic Nucleic Acid
ttccg

(19) United States
(12) Patent Application Publication
(10) Pub. No.: US 2024/0076653 A1
(43) Pub. Date: Mar. 7, 2024

(54) METHOD FOR CONSTRUCTING MULTIPLEX PCR LIBRARY FOR HIGH-THROUGHPUT TARGETED SEQUENCING

(71) Applicant: DONGKE ZHISHENG GENE TECHNOLOGY (BEIJING) CO. , LTD., Beijing (CN)

(72) Inventors: Jun ZHU, Beijing (CN); Bing BAI, Beijing (CN); Xin JIN, Beijing (CN)

(73) Assignee: MOKOBIO LIFE SCIENCE CORPORATION BEIJING, Beijing (CN)

(21) Appl. No.: 18/270,492

(22) PCT Filed: Dec. 31, 2021
(86) PCT No.: PCT/CN2021/143948

§ 371 Date: Jun. 30, 2023

(30) Foreign Application Priority Data
Dec. 31, 2020 (CN) 202011628234.2

(51) Int. Cl. C12N 15/10

(52) U.S. Cl. CPC C12N 15/1065

(57) ABSTRACT

A method for constructing a multiplex PCR library for high-throughput targeted sequencing: first acquiring a targeted DNA product by means of a high-specificity multiplex PCR reaction, and then digesting with a specific endonuclease, such that a specific molecular barcode is produced at the tail end of the PCR product; thus, the library construction process is more efficient, and the accuracy and sequencing depth of the obtained data are also ensured.

### CROSS REFERENCE TO THE RELATED APPLICATIONS

This application is the national phase entry of International Application No. PCT/CN2021/143948, filed on Dec. 31, 2021, which is based upon and claims priority to Chinese Patent Application No. 202011628234.2, filed on Dec. 31, 2020, the entire contents of which are incorporated herein by reference.

### SEQUENCE LISTING

The instant application contains a Sequence Listing which has been submitted in ASCII format via EFS-Web and is hereby incorporated by reference in its entirety. Said ASCII copy is named GBZD016_Sequence Listing.txt, created on 06/29/2023, and is 30,114 bytes in size.

### TECHNICAL FIELD

The present disclosure relates to the field of biological medicines, more specifically relates to a construction method of a DNA library, and in particular to a method for constructing a multiplex PCR library for high-throughput targeted sequencing.

### BACKGROUND ART

The present disclosure relates to the technical field of library construction, and in particular to a targeted high-throughput DNA library construction method. In the past decade, with the continuous advancement of new-generation sequencing technology, application of a life science research has been expanding. Different nucleic acid preparation methods and sequencing library construction methods are also more efficient.

High-throughput sequencing, i.e. next-generation sequencing (NGS), is a technology capable of achieving large-scale parallel sequencing on a high-density biochip, and has the characteristics of a high data yield and a low cost per amount of data. However, high-throughput sequencing has the disadvantage lying in a sequencing read; while a sequencing length is generally 2×300 bp or 2×150 bp. It may be very difficult to align and assemble obtained short-read sequencing sequences in a case without a reference genome or in a case of a genome including a sequence of a highly complex structure. At this time, a large-span large fragment library (mate pair library) may assist assembly of short sequences. In addition, the large fragment library is analyzed by the link algorithm, which may detect a structural variation such as insertion, deletion, inversion and aberration of a large fragment of a chromosome.

High-throughput targeted sequencing is a very cost-effective and highly sensitive detection means, and has a key link of targeted enrichment of target genes. At present, a main method for targeted enrichment includes a method for constructing a library based on hybrid capture and PCR. In general, the method based on hybrid capture is expensive and has tedious operation steps due to the use of magnetic beads coated with streptavidin, and requires more DNA specimens at the same time. With the development of technology in recent years, compared with hybrid capture, a targeted enrichment technology based on PCR using a unique molecular identifier (UMI) technology has made great progress, and may solve the original problem of difficult removal of PCR repetitive sequences; however, error in UMI is still difficult to eliminate, and the operation steps are tedious. Therefore, there is a need for providing an accurate, efficient, simple and convenient method for constructing a multiplex PCR targeted enrichment library.

Existing methods for constructing targeted enrichment libraries based on PCR mainly include AmpliSeq (thermo), SLIM Amplification, Relay PCR and the like. These methods all include two-step PCR reactions, that is, the first step is targeted amplification of a target fragment; and the second step is PCR enrichment after adaptor ligation. However, these methods all use traditional TA ligation or blunt end ligation; a non-specific amplification control link is not added in the whole library construction process; and a non-specific amplification product cannot be well removed either. This situation is particularly prominent in targeted methylation sequencing. Due to the vast majority of cytosine of DNA treated with bisulfite being changed into thymine, it is easy to form primer dimers or non-specific amplification between multiple primers.

### SUMMARY OF THE INVENTION

A purpose of the present disclosure is to provide a method for constructing a multiplex PCR library for high-throughput targeted sequencing.

In order to achieve above objective, the present disclosure employs the following technical means:

The present disclosure relates to a method for constructing a multiplex PCR library for high-throughput targeted sequencing. By adding polybasic MoCODE barcodes to a specific amplification product, and using the MoCODE barcodes to efficiently ligating the amplification product to sequencing adaptors comprising MoCODE barcode decoding sequences, a library is constructed; the MoCODE barcodes refer to overhanging single-stranded nucleotide sequences constituting two sticky ends of an obtained PCR product after the multiplex PCR product is digested with a specific endonuclease; and the MoCODE barcode decoding sequences are nucleotide sequences complementary to the MoCODE barcodes.

Preferably, a generation mode of the MoCODE barcodes comprises: one or more of a modified nucleotide, a nicking enzyme, an endonuclease, chemical modification, base photodegradation and the like; preferably, the modified nucleotide comprises one or more of dUTP, dITP and an RNA base.

Preferably, the MoCODE barcodes may be the same or different within molecules.

Preferably, the MoCODE barcodes are non-random specific barcodes.

Preferably, the MoCODE barcode has a length of 2-20 nt.

Preferably, the MoCODE barcode decoding sequence is complementary to a MoCODE barcode sequence, having a length of 2-20 nt.

Preferably, the sequencing adaptor may be artificially designed and synthesized, or matched with an own fragment sequence of a target segment.

Preferably, each sequencing adaptor may be a single adaptor and a bidirectional adaptor.

Preferably, enrichment in each specific segment may be decoded by virtue of the single adaptor, the bidirectional adaptor or automatic cyclization.

The present disclosure further relates to a primer for multiplex PCR for high-throughput targeted sequencing, the primer comprises a MoCODE barcode generating sequence; preferably, the primer comprises the sequences shown as Seq ID Nos: 1-22, 27-52, 53, 55, 57-104, 109 and 111.

Accordingly, the present disclosure further relates to a sequencing adaptor for multiplex PCR for high-throughput targeted sequencing, the sequencing adaptor comprises a MoCODE barcode decoding sequence; preferably, the sequencing adaptor further comprises one or more of a sequencing adaptor of a sequencing platform and an index label; preferably, the sequencing adaptor comprises a universal sequence for high-throughput sequencing, an index label and a MoCODE barcode decoding sequence; and the sequencing adaptor comprises the sequences shown as Seq ID Nos: 23-26, 54, 56, 105-108, 110 and 112.

The present disclosure relates to a method for constructing a multiplex PCR library for high-throughput targeted sequencing, compriseing the following steps:
1) extracting DNA from a to-be-tested specimen;
2) performing a multiplex PCR reaction, wherein each primer, participating to the multiplex PCR reaction, comprises a specific MoCODE barcode generating sequence; preferably, the primer further comprises a gene-specific sequence;
3) purifying a PCR product obtained in step 2) with magnetic beads;
4) making the PCR product purified in step 3) generate a 5′ sticky end and a 3′ sticky end, and generating MoCODE barcodes at the 5′ sticky end and the 3′ sticky end respectively;
5) purifying the PCR product comprising the MoCODE barcodes in step 4) with the magnetic beads;
6) ligating the PCR product, comprising the MoCODE barcodes, purified in step 5) to the sequencing adaptors, wherein the sequencing adaptor comprising MoCODE barcode decoding sequences complementary to the MoCODE barcodes;
7) purifying a ligation product obtained in step 6) with the magnetic beads, and completing construction of the multiplex PCR library for high-throughput targeted sequencing.

Preferably, a generation mode of the MoCODE barcodes in step 4) comprises: one or more of a modified nucleotide, a nicking enzyme, an endonuclease, chemical modification, base photodegradation and the like; preferably, the modified nucleotide comprises one or more of dUTP, dITP and an RNA base; more preferably, the generation mode of the MoCODE barcodes is to use a specific endonuclease for digestion.

Preferably, in step 4), one MoCODE barcode is generated at each of the 5′ sticky end and the 3′ sticky end, wherein the MoCODE barcodes at the 5′ sticky end and the 3′ sticky end may be same or different.

Preferably, in step 6), each sequencing adaptor may be a single adaptor, a bidirectional adaptor or a cyclization adaptor.

Compared with the prior art, the present disclosure has the following advantages:

(1) Reduction in an Amount of a Non-Specific Product in Multiplex PCR Amplification

Although unique molecular identifiers (UMIs) are introduced into a method for constructing a library based on PCR targeted enrichment at present, errors in the library construction and sequencing process may be filtered to a certain degree; however, random errors are not only caused by a sequence of a template fragment, but may also be from sequences of the UMIs own. If the errors are from the UMIs, PCR repetitive sequences may be wrongly recognized as being from unique molecules identified by the UMIs, which may cause overestimation in a sequencing depth, and then affects the sequencing quality. As random sequences intrinsically, the UMIs cannot remove the non-specific amplification product, a primer dimer, or a more complex single-stranded or double-stranded multimer in the multiplex PCR.

By designing a highly specific multiplex PCR primer sets, and adding a particular digestion site and a unique particular sequence to each set of primers, only after being subjected to digestion, a correctly amplified PCR product can be ligated to a specifically paired adaptor, thereby constructing the sequencing library. A dimer and a multimer generated in the amplification process are removed by digestion with the specific endonuclease. As the non-specific amplification product cannot be correctly combined with a decoding adaptor, a final ligation product cannot be amplified and recognized in the high-through sequencing process; and all or the vast majority of sequencing data is specific target fragment, which greatly increase a hit rate of the sequencing data, so as to ensure a sequencing depth.

(2) High Efficiency and Reduction in Pollution

By designing sticky end adaptor ligation, compared with the effect of only ligase existing in blunt end ligation, the complementary effect of bases is more highlighted; and the affinity of the enzyme and a substrate is improved at the same time, thereby remarkably improving the ligation efficiency. Compared with two PCRs in methods for constructing targeted enrichment libraries based on PCR from other companies, the whole library construction process only require one-step PCR reaction, which reduces pollution and provides stronger pollution resistance.

(3) Simple and Convenient Operation, and Shortening of Time

By designing the highly specific multiplex PCR primer sets, and improving the adaptor ligation efficiency, the library construction process becomes more efficient; and compared with the methods for constructing the targeted enrichment libraries based on the PCR from other companies, a manual operation time is shortened by 40-50%, and the overall library construction time is shortened by 30-40%.

### BRIEF DESCRIPTION OF THE DRAWINGS

FIG. 1 is a diagram showing a process of constructing libraries using different MoCODEs in a method of the present disclosure;

FIG. 2 is a structural schematic diagram of a forward primer and a reverse primer of multiplex PCR of the present disclosure;

FIG. 3 is a structural schematic diagram of a forward adaptor and a reverse adaptor of the present disclosure;

FIG. 4A is a structural schematic diagram of a double-stranded structure with MoCODEs (different) at two ends of a PCR product in embodiment 3 of the present disclosure;

FIG. 4B is a structural schematic diagram of a double-stranded structure of a forward adaptor in embodiment 3 of the present disclosure;

FIG. 4C is a structural schematic diagram of a double-stranded structure of a reverse adaptor in embodiment 3 of the present disclosure;

FIG. 5A is a structural schematic diagram of a double-stranded structure with MoCODEs (same) at two ends of a PCR product in embodiment 4 of the present disclosure;

FIG. 5B is a structural schematic diagram of a double-stranded structure of a forward adaptor in embodiment 4 of the present disclosure;

FIG. 5C is a structural schematic diagram of a double-stranded structure of a reverse adaptor in embodiment 4 of the present disclosure;

FIG. 6A is a schematic diagram of a primer used when a MoCODE barcode is generated by amplifying an own MoCODE generating sequence included in a target segment the present disclosure;

FIG. 6B is a schematic diagram of a PCR amplified target fragment comprising a MoCODE generating sequence own, which is used when a MoCODE barcode is generated by amplifying own MoCODE generating sequence included in a target segment the present disclosure;

FIG. 6C is a schematic diagram of a PCR product in which a MoCODE barcode is generated when a MoCODE barcode is generated by amplifying own MoCODE generating sequence included in a target segment the present disclosure;

FIG. 7 is a diagram showing agarose gel electrophoresis results of a PCR amplification product in embodiment 1 of the present disclosure;

FIG. 8 is a diagram showing agarose gel electrophoresis results of a product of sequencing adaptor ligation in embodiment 2 of the present disclosure.

### DETAILED DESCRIPTION OF THE INVENTION

According to the above contents of the present disclosure, various modifications, substitutions or variations may further be made without departing from the basic technical concept above of the present disclosure according to the common technical knowledge and conventional means in the art.

I. Definition

The term “sample” includes a specimen or a culture (for example, a microbiological culture) including nucleic acids, and is further intended to include a biological sample and an environmental sample. The sample may include a specimen of synthetic origin. The biological sample includes whole blood, a serum, plasma, umbilical cord blood, chorionic villi, an amniotic fluid, a cerebrospinal fluid, a spinal fluid, a lavage fluid (for example, a bronchoalveolar lavage fluid, a gastric lavage fluid, a peritoneal lavage fluid, a catheter lavage fluid, an ear lavage fluid and an arthroscopic lavage fluid), a biopsy sample, urine, feces, sputum, saliva, nasal mucus, a prostatic fluid, semen, lymph, bile, tears, sweat, milk, a breast fluid, embryonic cells and fetal cells. In a preferred embodiment, the biological sample is the blood, more preferably, the plasma. As used herein, the term “blood” includes the whole blood or any blood fraction, such as the serum and the plasma as conventionally defined. The blood plasma refers to a whole blood fraction generated by centrifuging the blood treated with an anticoagulant. The blood serum refers to a water sample portion of a fluid remained after the blood sample is solidified. The environmental sample includes an environmental material, such as a surface substance sample, a soil sample, a water sample and an industrial sample, as well as a sample obtained from food and dairy product processing apparatuses, instruments, devices and appliances, disposable articles and non-disposable articles. These examples should not be interpreted as limiting types of sample that may be applied to the present invention.

The terms “target”, “target nucleic acid” and “target gene” are intended to refer to any molecule to be detected or measured in existence, or to be detected researched in a function, interaction, or characteristics.

The terms “nucleic acid” and “nucleic acid molecule” may be used interchangeably throughout the present disclosure. The terms refer to an oligonucleotide, an oligomer, a polynucleotide, deoxyribonucleotide (DNA), genomic DNA, mitochondrial DNA (mtDNA), complementary DNA (cDNA), bacterial DNA, viral DNA, viral RNA, RNA, messenger RNA (mRNA), transfer RNA (tRNA), ribosomal RNA (rRNA), siRNA, catalytic RNA, cloning, a plasmid, M13, P1, a clay, a bacterial artificial chromosome (BAC), a yeast artificial chromosome (YAC), an amplified nucleic acid, an amplicon, a PCR product and other types of amplified nucleic acids, RNA/DNA hybrids and polyamide nucleic acid (PNA). All of these nucleic acids and nucleic acid molecules may be in a single-stranded or double-stranded form, and unless otherwise restricted, may include known analogues of natural nucleotides that may function in a manner similar to naturally occurring nucleotides, and their combinations and/or mixtures. Therefore, the term “nucleotide” refers to a naturally occurring and modified/non-naturally occurring nucleotide, including nucleoside triphosphate, nucleoside diphosphate, nucleoside monophosphate, and a monophosphate monomer existing in a polynucleic acid or the oligonucleotide. The nucleotide may further be ribose, 2′-deoxy, 2′, 3′-deoxy and a great amount of other nucleotide analogues well known in the art. The analogues include chain-terminating nucleotides, such as 3′-O-methyl, halogenated base or sugar substitutions; alternative sugar structures including non-sugar, alkyl ring structure; alternative bases including inosine; denitrification modifications; chi and psi, adaptor modifications; mass label modifications; phosphodiester modifications or replacements, including phosphorothioate, methylphosphonate, boranophosphate, amides, esters and ethers; and substantial or complete internucleotide substitutions, including cleavage ligation, such as photocleavable nitrophenyl portions.

The term “amplification reaction” refers to any in vitro mode of copying for amplifying a target nucleic acid sequence. “Amplification” refers to a step making a solution be under the condition sufficient to allow amplification. Components in the amplification reaction may include, but are not limited to, primers, polynucleotide templates, polymerases, nucleotides, dNTPs and the like. The term “amplification” generally refers to an “exponential” increase in target nucleic acids. However, “amplification” as used herein may also refer to linear increase in a number of appointed target nucleic acid sequences, but it is different from the one-time single primer extension step.

The term “polymerase chain reaction” or “PCR reaction” refers to a method for amplifying a specific segment or subsequence of target double-stranded DNA by geometric progression. The PCR is well known by those skilled in the art.

The term “oligonucleotide” refers to a linear oligomer of natural or modified nucleoside monomers ligated by virtue of a phosphodiester bond or its analogues. The oligonucleotides include deoxyribonucleosides, ribonucleosides, end-capped isomer forms thereof, peptide nucleic acids (PNA) and the like, which can specifically bind to the target nucleic acids. In general, monomers are ligated by virtue of the phosphodiester bonds or their analogues to form the oligonucleotides ranging from several monomeric units (for example, 3-4) to dozens of monomeric units (for example, 40-60) in size. Every time the oligonucleotides are expressed by alphabetical sequences (such as “ATGCCTG”), it should be understood that, unless otherwise noted, the nucleotides are in an order from 5′ to 3′ from left to right. “A” refers to deoxyadenosine; “C” refers to deoxycytidine; “G” refers to deoxyguanosine; “T” refers to deoxythymidine; and “U” refers to ribonucleoside and uridine. In general, the oligonucleotides includ four natural deoxynucleotides; however, they may further include ribonucleoside or non-natural nucleotide analogues. In a case that the enzymes have requirements for particular oligonucleotide or polynucleotide substrates for activity (for example, single-stranded DNA and RNA/DNA duplexes), a choice of appropriate composition of the oligonucleotide or polynucleotide substrates is completely within the knowledge of ordinary skilled in the art.

The term “primer”, i.e. “oligonucleotide primer”, refers to a polynucleotide sequence, which is hybridized with a sequence on a target nucleic acid template and promotes detection of an oligonucleotide probe. In the amplification embodiment of the present invention, the oligonucleotide primers serve as starting points of synthesis of the nucleic acids. In the non-amplification embodiment, the oligonucleotide primers may be used for creating structures which can be cleaved by a cleavage reagent. Each primer may have a plurality of lengths, and has usually less than 50 nucleotides in length. The length and sequence of each primer used in the PCR may be designed based on the principle known by those skilled in the art.

“Mismatched nucleotide” or “mismatch” refers to nucleotides which are not complementary to the target sequence at one or more positions. Each oligonucleotide probe may have at least one mismatch, but may also have 2, 3, 4, 5, 6, 7 or more mismatched nucleotides.

The term “specific” or “specificity” for binding a molecule to another molecule (such as a probe for a target polynucleotide) refers to recognition, contact and stable complex formation between the two molecules, as well as remarkably reduced recognition, contact or formation of complexes between the molecule and other molecules. The term “annealing” as used herein refers to formation of the stable complex between two molecules.

The term “cleavage reagent” refers to any tool capable of cleaving the oligonucleotides to produce fragments, including, but not limited to, enzymes. For the methods, in which amplification does not occur, the cleavage reagent may be used only for cleaving, degrading, or otherwise separating a second portion of the oligonucleotide probe or a fragment thereof. The cleavage reagent may be an enzyme. The cleavage reagent may be natural, synthetic, unmodified or modified.

For the method in which amplification occurs, the cleavage reagent is preferably an enzyme having the synthetic (or polymerization) activity and nuclease activity. Such enzyme is generally a nucleic acid amplification enzyme. An example of the nucleic acid amplification enzyme is a nucleic acid polymerase such as Thermus aquaticus (Taq), a DNA polymerase (TaqMan®), or an Escherichia coli (E. coli) DNA polymerase I. The enzyme may be natural, synthetic, unmodified or modified.

The term “nucleic acid polymerase” refers to an enzyme for catalyzing the nucleotide to incorporate into the nucleic acid. An exemplary nucleic acid polymerase includes a DNA polymerase, an RNA polymerase, a terminal transferase, a reverse transcriptase, a telomerase and the like.

“Thermostable DNA polymerase” refers to such DNA polymerase that if it withstands a high temperature with in a selected time period, it is stable (that is, resistant to decomposition or denaturation) and retains sufficient catalytic activity. For example, if the thermostable DNA polymerase withstands the high temperature for a time necessary for double-stranded nucleic acid denaturation, the thermostable DNA polymerase retains sufficient activity to achieve a subsequent primer extension reaction. The heating conditions necessary for nucleic acid denaturation are well known in the art, and exemplified in U.S. Pat. Nos. 4,683,202 and 4,683,195. The thermostable polymerase as used herein is usually suitable for a temperature cycling reaction such as the polymerase chain reaction (“PCR”). An example of the thermostable polymerase includes the Thermos aquaticus (Taq), the DNA polymerase (TaqMan®), a Thermus species Z05 polymerase, a Thermus flavus polymerase, a Thermotoga maritima polymerase such as TMA-25 and TMA-30 polymerases, a Tth DNA polymerase and the like.

“Modified polymerase” refers to a polymerase having at least one monomer different from a reference sequence such as a natural or wild-type form of the polymerase or another modified form of the polymerase. An exemplary modification includes monomer insertion, deletion or substitution. The modified polymerase further includes a chimeric polymerase having identifiable component sequences (for example, a structural or functional domain) derived from two or more parents. The definition of the modified polymerase further includes chemically modified polymerases including the reference sequence. An Example of the modified polymerase includes a G46E E678G CS5 DNA polymerase, a G46EL329A E678G CS5 DNA polymerase, a G46E L329A D640G S671F CS5 DNA polymerase, a G46E L329AD640G S671F E678G CS5 DNA polymerase, a G46E E678G CS6 DNA polymerase, a Z05 DNA polymerase, a ΔZ05 polymerase, a ΔZ05-Gold polymerase, a ΔZ05R polymerase, an E615G Taq DNA polymerase, an E678G TMA-25 polymerase, an E678G TMA-30 polymerase and the like.

The term “5′ to 3′ nuclease activity” or “5′-3′ nuclease activity” refers to the activity of the nucleic acid polymerase which is generally related to synthesis of a nucleic acid chain, so as to remove the nucleotide from the 5′ end of the nucleic acid chain. For example, the Escherichia coli DNA polymerase I has the activity, while a Klenow fragment does not have the same. Some enzymes having the 5′ to 3′ nuclease activity are 5′ to 3′ exonucleases. Examples of such 5′ to 3 exonucleases include: an exonuclease from B. subtilis, a phosphodiesterase from a spleen, a exonuclease, an exonuclease II from a yeast, an exonuclease V from the yeast, and an exonuclease from Neurospora crassa.

The terms “MoCODE barcode”, “Molecular code” and “specific molecular barcode” used herein refer to overhanding single-stranded sequences of the two sticky ends of an obtained PCR product after a multiplex PCR product is digested with a specific endonuclease.

The term “MoCODE barcode decoding sequence” or “molecular barcode decoding sequence” used herein is a nucleotide sequence complementary to the “MoCODE barcode”, “Molecular code” and “specific molecular barcode”.

### II. Embodiments

A method for constructing a multiplex PCR library for high-throughput sequencing of the present disclosure is based on the following principle:
1. A MoCODE barcode (molecular code) was introduced into a primer of each amplified segment.
2. MoCODE barcodes of each pair of amplification primers may be different or same.
   Specific amplification products were selected by virtue of matching during later adaptor ligation. Each MoCODE barcode may have a length of 2 nt-20 nt or longer.
3. As not being effectively matched with the adaptors, non-specific fragments cannot form a correct structure required for sequencing, cannot be amplified in a sequencing reaction system, and thus cannot be removed from the reaction system.
4. Compared with TA ligation or blunt end ligation for current library construction, matching ligation between the MoCODE barcodes and the adaptors is sticky end ligation which may improve the ligation efficiency and final detection sensitivity.
5. Amplification: gene-specific and universal amplification and MoCODE barcode introduction may be achieved in a same PCR reaction, which shortens operation steps and manual operation time, avoids cross pollution in library construction, reduce the cost, and improve the clinical practicality.
6. The MoCODE barcodes may be used matching with UMIs, and the mutation detection accuracy of targeted sequencing is further improved by virtue of error correction.

In the method for constructing the multiplex PCR library for high-throughput targeted sequencing of the present disclosure, by adding the MoCODE barcodes to the specific amplification product, and using the matched sequencing adaptors comprising the MoCODE barcode decoding sequence for efficient ligation, the library is constructed.

In some embodiments of the present disclosure, specimen sources of the specific amplified product include, but are not limited to, genomic DNA, free DNA, free cells, cDNA generated by reverse transcription of RNA specimens and the like.

In some embodiments of the present disclosure, template DNA for the multiplex PCR reaction may be DNA, bisulfite-transformed DNA, cDNA and the like.

In some embodiments of the present disclosure, an extraction method of the template DNA for the multiplex PCR reaction may be a column extraction method, a magnetic bead method, phenol-chloroform extraction-ethanol or isopropanol precipitation, and the like.

In some embodiments of the present disclosure, each primer, participating to the multiplex PCR reaction, comprises a specific MoCODE barcode generating sequence; preferably, the primer further comprises a gene-specific sequence.

In some embodiments of the present disclosure, a generation mode of the MoCODE barcodes comprises: a modified nucleotide (dUTP, dITP or RNA base), a nicking enzyme, an endonuclease, chemical modification, base photodegradation and the like. Its purpose is to perform recognizable site cleavage at ends of the PCR product, so as to obtain the sticky ends comprising the MoCODE barcodes.

In a specific embodiment of the present disclosure, the generation mode of the MoCODE barcodes is that: each primers for the multiplex PCR reaction might further comprises a universal recognition site of a specific endonuclease between primers at the 5′ end, in addition to a gene-specific sequence, and then a purified PCR product was digested with the specific endonuclease (one or two). The digested PCR product would include two sticky ends. An overhanding single-stranded sequence of each sticky end formed a specific molecular barcode, i.e. Molecular CODE (MoCODE) barcode.

In some embodiments of the present disclosure, each primer comprises the sequences shown as Seq ID Nos: 1-22, 27-52, 53, 55, 57-104, 109 and 111, wherein n represents a nucleotide dITP or dUTP.

In some embodiments of the present disclosure, the generation mode of each MoCODE barcode is that: in addition to a gene-specific sequence, each primers for the multiplex PCR reaction might further comprise a dITP site where might form a sticky end having 6 bases after digestion recognition with a specific enzyme, and then a MoCODE barcode sequence was generated.

In some embodiments of the present disclosure, the MoCODE barcodes may be the same or different in molecules. For example, “same” represents that the MoCODE barcodes at the two ends of a molecule of one PCR product are formed by cleavage after being recognized with one endonuclease; and “different” represents the MoCODE barcodes at the two ends of the molecule of one PCR product are formed by cleavage after being recognized with different endonucleases.

In some embodiments of the present disclosure, one nucleotide molecule includes one kind of MoCODE barcodes, for example, the MoCODE barcodes generated at the 5′ and 3′ sticky ends of the molecule of one PCR product are the same.

In some embodiments of the present disclosure, one nucleotide molecule includes two kinds of MoCODE barcodes, for example, the MoCODE barcodes generated at the 5′ and 3′ sticky ends of the molecule of one PCR product are different.

In some embodiments of the present disclosure, the MoCODE barcodes are non-random specific barcodes.

In some embodiments of the present disclosure, each MoCODE barcode has a length of 2-20 nt.

In some embodiments of the present disclosure, each MoCODE barcode comprises the sequences shown as Seq ID Nos: 53, 59, 109 and 111.

In some embodiments of the present disclosure, each MoCODE barcode decoding sequence is complementary to a MoCODE barcode sequence, having a length of 2-20 nt.

In some embodiments of the present disclosure, each MoCODE barcode decoding sequence comprises the sequences shown as Seq ID Nos: 54, 56 110 and 112.

In some embodiments of the present disclosure, each sequencing adaptor comprising the MoCODE barcode decoding sequence may be artificially designed and synthesized, or matched with an own fragment sequence of a target segment.

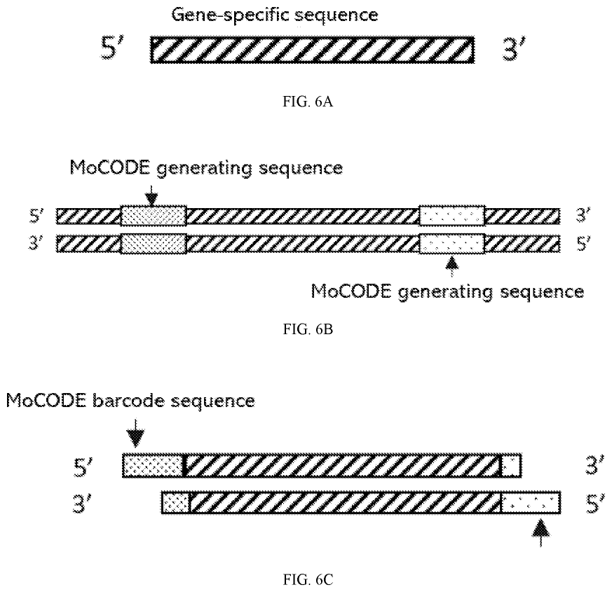

Each sequencing adaptor including the MoCODE barcode decoding sequence may be matched with the own fragment sequence of the target segment is illustrated as follows: if the PCR amplified target segment includes the MoCODE generating sequence intrinsically, and the intrinsically included MoCODE generating sequence would be used for generating the MoCODE barcode at the 5′ end, there is no need for the primer at the 5′ end of the PCR carrying the MoCODE generating sequence; and if MoCODE intrinsically included in the PCR amplified target segment would be used for generating the MoCODE barcode at the 3′ end, there is no need for the primer at the 3′ end of the PCR carrying the MoCODE generating sequence (FIG. 6A).

In some embodiments of the present disclosure, each sequencing adaptor comprises the sequences shown as Seq ID Nos: 23-26 and 105-108, wherein “nnnnnnnn”, [i5] or [i7] represents an index label, for example, an Illumina Index label sequence of 8 nt. As well known in the art, the 5′ end for sticky ligation may be phosphorylated.

In some embodiments of the present disclosure, in the primer comprises sequences shown as Seq ID Nos: 57-104, “n” or “I” at position 5 is “dITP”.

In some embodiments of the present disclosure, a PCR amplified target fragment may comprise one or two own MoCODE generating sequences (FIG. 6B). Accordingly, the own MoCODE generating sequences may be used for generating MoCODE barcodes at one end or two ends of a DNA molecule. Through digestion with the endonuclease corresponding to the own MoCODE generating sequences, corresponding MoCODE barcodes may be generated at one end or two ends of the PCR product (FIG. 6C).

In some embodiments of the present disclosure, each sequencing adaptor including the MoCODE barcode decoding sequence may be a single adaptor or a bidirectional adaptor; and enrichment in each specific segment may be decoded by virtue of the single adaptor, the bidirectional adaptor or automatic cyclization. The “single adaptor” is used in a case that the MoCODE barcodes are the two ends of the PCR product are the “same”; the “bidirectional adaptor” is used in a case that the MoCODE barcodes are the two ends of the PCR product are “different”. It is to be understood that in the case that different adaptors are used, if the adaptors on two sides of the non-specific product are the same, a correct sequenced product cannot be formed, thereby removing the non-specific product in a sequencing link.

In some embodiments of the present disclosure, “cyclization” may use various MoCODE barcodes, having a structure of MoCODE, a common sequence combined by sequencing primers and a gene-specific sequence. The cyclization decoding step is as follows: PCR, digestion, circularization, exonuclease digestion, and add-on PCR (addition of complete sequencing primer binding site, library index and sequences adapter), which may be used for forming various amplicons.

In some embodiments of the present disclosure, the sequencing adaptors comprising the MoCODE barcode decoding sequences include a forward sequencing adaptor and a reverse sequencing adaptor. The forward sequencing adaptor includes a MoCODE barcode decoding sequence complementary to the MoCODE barcode at the 5′ end of the digested PCR product; and the reverse sequencing adaptor comprises a MoCODE barcode decoding sequence complementary to the MoCODE barcode at the 3′ end of the digested PCR product.

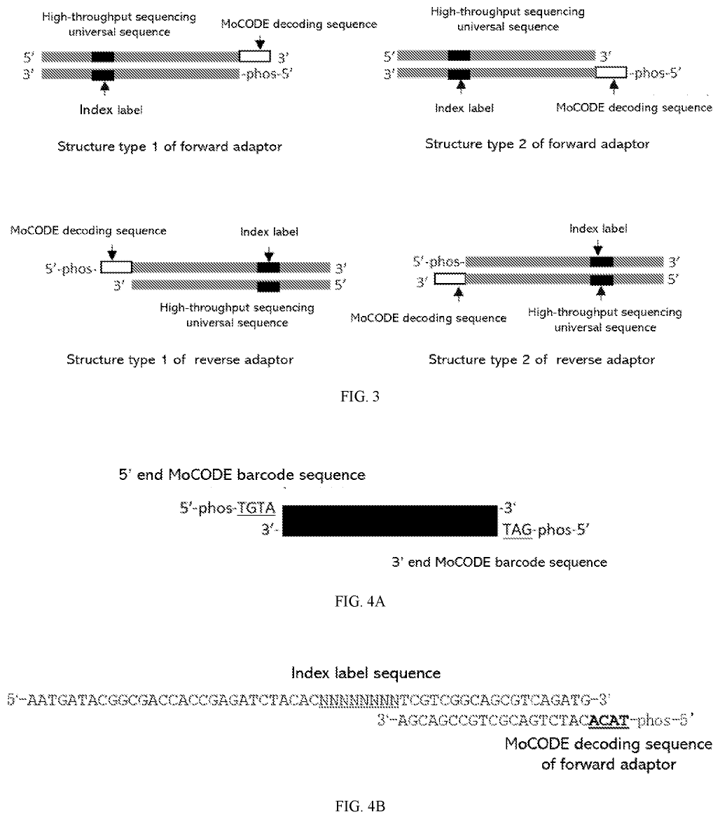

Also, the forward sequencing adaptor and the reverse sequencing adaptor further include an adaptor upper chain and an adaptor lower chain respectively. The adaptor upper chain is a sense chain; and the adaptor lower chain is an antisense chain. The MoCODE barcode decoding sequence may be located at the 3′ end of the adaptor upper chain of the forward sequencing adaptor, or at the 5′ end of the adaptor lower chain of the reverse sequencing adaptor, or at the 5′ end of the adaptor upper chain of the reverse sequencing adaptor or at the 3′ end of the adaptor lower chain of the reverse sequencing adaptor (FIG. 3).

In some embodiments of the present disclosure, multiplex amplification of 2-1000 target segments may be achieved. Each target segment may have its own specific barcode; and a plurality of target segments may share one barcode.

In some embodiments of the present disclosure, the MoCODE barcodes are non-random specific barcodes, and may further be used for multi-target-segment cancatmerization.

In some embodiments of the present disclosure, a DNA polymerase used in multiplex PCR may be a Taq polymerase, PFx, KOD, Pfu, Q5, Bst, Phusion and other commercial enzymes.

In some embodiments of the present disclosure, a ligase used in multiplex PCR may be a T4 DNA ligase, a 9 NTM DNA ligase, aTaq DNA ligase, a Tth DNA ligase, aTfiDNA ligase, Ampligase R and the like.

In some embodiments of the present disclosure, excessive removal of the sequencing adaptor may be achieved by the magnetic bead method, the column extraction method, the ethanol precipitation method, an agarose or polyacrylamide gel recovery method and the like.

In some embodiments of the present disclosure, the constructed library is suitable for high-throughput sequencing platforms such as Illumina, Roche, ThermoFisher, Pacific Biosciences, Beijing Genomics Institute, Oxford Nanopore Technologies, Huayinkang, and Hanhai Gene.

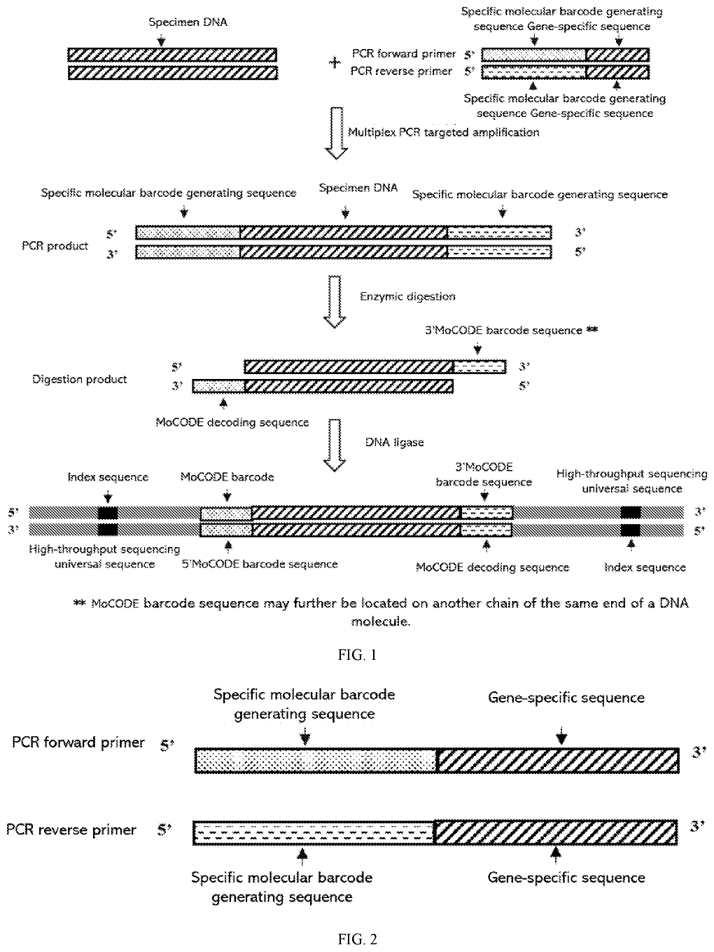

Particularly, in some embodiments of the present disclosure, the method for constructing the multiplex PCR library for high-throughput targeted sequencing comprises the following steps (an example library construction process is shown in FIG. 1):
Step 1: DNA was extracted from a to-be-tested specimen; and if it was methylation sequencing, library construction required subsequent transformation with bisulfite.
Step 2: with a DNA specimen treated in step 1 as a template, multiplex PCR reaction was performed with a high-fidelity PCR enzyme and multiple pairs of primers (FIG. 2), wherein each pair of primers, participating to the multiplex PCR reaction, comprises a universal specific molecular barcode generating sequence between primers at the 5′ end, in addition to a gene-specific sequence.
Step 3: a PCR product was purified with magnetic beads.
Step 4: the purified PCR product in step 3 was digested with a specific endonuclease. Each of the 3′ end and 5′ end of the correctly amplified multiplex PCR product should include a specific barcode generation site. After digestion with the specific endonucleases, a sticky ends may be formed, that is, the MoCODE barcode sequences are generated to mediate the ligation of step 5. There are many generation modes of the MoCODE barcodes, comprising: a modified nucleotide (dUTP, dITP or RNA base), a nicking enzyme, an endonuclease, chemical modification, base photodegradation and the like.
Step 5: a digestion product in step 4 was purified with the magnetic beads.
Step 6: a forward sequencing adaptor and a reverse sequencing adaptor were introduced into the digestion product purified in step 5 using a ligase capable of catalyzing ligation between the sticky ends. The introduced forward sequencing adaptor comprises a universal sequence (which may comprise an index label sequence) for high-throughput sequencing, and a MoCODE barcode decoding sequence complementary to the MoCODE at the 5′ end of the digestion PCR product obtained in step 4. The introduced reverse sequencing adaptor comprises a universal sequence (which comprises an index label sequence) for high-throughput sequencing, and a MoCODE barcode decoding sequence complementary to the MoCODE at the 3′ end of the digestion PCR product obtained in step 4 (FIG. 3).
Step 7: a ligation product was purified with the magnetic beads, and the sequencing library was constructed.

### III. EXAMPLES

The following further describes the present invention in combination with specific examples; and the advantages and the characteristics of the present invention will be clearer with the description. However, these examples are only exemplary, and should not be construed as limiting the present invention. Those skilled in the art should appreciate that modifications and substitutions could be made on details and forms without departing from the spirit and scope of the present invention, but all fall within the scope of protection of the present invention.

### Example 1: Elimination of Non-Specific PCR Product with Targeted Methylation PCR Enrichment Using MoCODE

In this example, 10 pairs of bisulfite sequencing primers (BSP) in 2 sets were designed, and each primer in the 2 sets include a same gene-specific sequence, wherein in an experimental group, each pair of BSPs include universal specific molecular (MoCODE) barcode generating sequences between primers at 5′ ends, in addition to a gene-specific sequence; and in a control group, each pair of BSPs include the gene-specific sequences only, and do not include the specific molecular (MoCODE) barcode generating sequences at the 5′ ends. Two MoCODE barcode sequences were generated by digesting PCR products with two restricted endonucleases. Then, the enrichment effects of the two groups of products were observed by virtue of agarose gel electrophoresis.

1) Preparation of PCR Template
a) Genomic DNA of Hela cells (America NEB Company) was subjected to bisulfite transformation with an EZ DNA Methylation-Gold Kit (America ZYMO Company).
b) A concentration of the transformed DNA was measured using a Qubit fluorometer.
c) A concentration of bisulfite-transformed DNA was adjusted to 50 ng/μl with water.

2) Multiplex PCR
a) PCR reaction system

| Component | Volume | |
|---|---|---|
| Nuclease-free water | 21.5 | μl |
| 2 × KOD-Multi Epi PCR premixed solution | 25 | μl |
| Primer mixed solution (10 μM) | 1.5 | μl |
| Genomic DNA, treated with sulfite, of Hela cells | 1 μl (50 ng) | |
| KOD-Multi & Ep (TOYOBO) | 1 | μl |
| Total volume | 50 | μl |

b) PCR program Step 1:94° C., 2 min.
Step 2: 6 cycles (98° C., 10 s; 59° C., 5 s; 68° C., 5s).
Step 3: 35 cycles (98° C., 10 s; 68° C., 10s).
Step 4: 68° C., 1 min.
Step 5: keeping at 8° C.

3) A Multiplex PCR Product was Purified with HiPrep PCR Magnetic Beads (America NEB Company)
a) The PCR product was purified with 60 μl of magnetic beads (1.2 times).
b) The purified product was eluted in 15 μl of water.
c) A concentration of the purified PCR product was measured using the Qubit fluorometer.
d) A concentration of the product was adjusted to 10 ng/μl with water.

4) The purified PCR product was treated with restricted endonucleases Bbvl and Earl (the structural schematic diagram of the generated product is shown as FIG. 5A).

| Component | Volume | |
|---|---|---|
| 10 × Cutsmart buffer solution (NEB) | 2 | μl |
| BbvI (NEB, 2 U/μl) | 1 | μl |
| EarI (NEB, 20 U/μl) | 0.5 | μl |
| Purified PCR product | 5 μl 50 ng | |
| Nuclease-free water | 11.5 | μl |
| Total volume | 20 | μl |

The product was incubated on a thermocycler for 30 min at 37° C.

A resultant was incubated for 20 min at 65° C., to make the enzymes lose activity.

A reaction mixed solution was purified using HiPrep PCR magnetic beads (1.2×), and eluted in 15 μl of water.

5) Agarose Gel Electrophoresis
a) 2% agarose gel was prepared with 0.5×TBE, and a nucleic acid dye (GelSafe) was added (1 μl of dye per 10 ml of system).
b) The purified PCR product was treated with 5 μL of restricted endonuclease.
c) 150 V electrophoresis was performed for 30 minutes, and the product was photographed with a gel imaging system for observation.

6) Results of Agarose Gel Electrophoresis

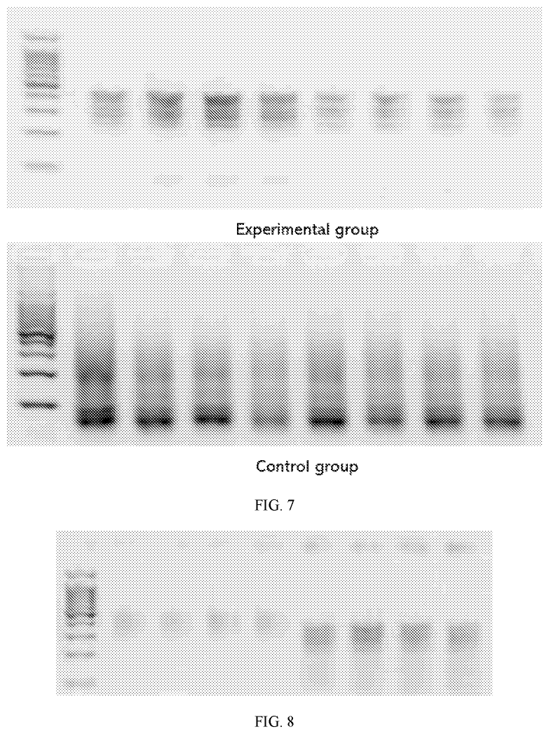

In the experimental group, it can be seen that the PCR amplification product with 10 pairs of primers is clear in band without generation of a primer dimer. In the control group, the PCR product is in a smear shape, and there is an obvious primer dimer (FIG. 7).

7) PCR Primer Sequences Used in this Example

As shown in following, the forward primer and the reverse primer include universal specific molecular barcode generating sequences shown as Seq ID Nos: 1 and 12 respectively. The Moko 1-10 forward primer includes sequences shown as Seq ID Nos: 2-11, and the Moko1-10 reverse primer includes sequences shown as Seq ID Nos: 13-22.

| Name | Forward primer (5′ > 3′) | Reverse primer (5′ > 3′) |
|---|---|---|
| Universal specific | AGATCGGCAGCGTCAGATGTGTAT | AGATCGCTCTTCCGATCT |
| molecular barcode | AAGAGACAG (Seq ID No: 1) | (Seq ID No: 12) |
| generating sequence | | |
| (5′ > 3′) | | |
| MOKO1 | AGATCGGCAGCGTCAGATGTGTAT | AGATCGCTCTTCCGATCTATATAT |
| | AAGAGACAGGAGTAGTTGGGATTA | ATCAAACACTRGACTTAAAAT |
| | TAGGTGT (Seq ID No: 2) | (Seq ID No: 13) |
| MOKO2 | AGATCGGCAGCGTCAGATGTGTAT | AGATCGCTCTTCCGATCTCCTTA |
| | AAGAGACAGTTAGAAATTTAGTTG | AAACAAACTTATCTTCTCC (Seq |
| | TAGAGGGGG (Seq ID No: 3) | ID No: 14) |
| MOKO3 | AGATCGGCAGCGTCAGATGTGTAT | AGATCGCTCTTCCGATCTCACCT |
| | AAGAGACAGGAGGTTAGGGTTTTA | TAACAAATAAAATAATAATTCAC |
| | GATTGGGA (Seq ID No: 4) | (Seq ID No: 15) |
| MOKO4 | AGATCGGCAGCGTCAGATGTGTAT | AGATCGCTCTTCCGATCTTATAC |
| | AAGAGACAGGTAAYGAATTGGTAG | TAACTCCCTTCAACCATTA (Seq |
| | AGTTTTA (Seq ID No: 5) | ID No: 16) |
| MOKO5 | AGATCGGCAGCGTCAGATGTGTAT | AGATCGCTCTTCCGATCTCTACC |
| | AAGAGACAGTGAGGGTAAGAATTA | CACACCTACCAAACCTAA (Seq |
| | TTTAGAGGT (Seq ID No: 6) | ID No: 17) |
| MOKO6 | AGATCGGCAGCGTCAGATGTGTAT | AGATCGCTCTTCCGATCTATCAA |
| | AAGAGACAGAGGGTTAAAGAAGA | AAATAATTCTAAAAATATACA |
| | GAATGATTTAT (Seq ID No: 7) | (Seq ID No: 18) |
| MOKO7 | AGATCGGCAGCGTCAGATGTGTAT | AGATCGCTCTTCCGATCTACCAA |
| | AAGAGACAGGAGGGTTGAATATTA | CTTCTATATAACTAATAAATACAC |
| | AAAATAGTAGGGT (Seq ID No: 8) | A (Seq ID No: 19) |
| MOKO8 | AGATCGGCAGCGTCAGATGTGTAT | AGATCGCTCTTCCGATCTAAAAT |
| | AAGAGACAGGGATAATTATAAGAA | TCACTTCTAAATTTAAACCA (Seq |
| | TTGTAAAGGAGGAT (Seq ID No: 9) | ID No: 20) |
| MOK09 | AGATCGGCAGCGTCAGATGTGTAT | AGATCGCTCTTCCGATCTAAAAT |
| | AAGAGACAGGGTAGTTGGAAATG | AATCTTCATCAAATTAATAAAAA |
| | GTAAATTTGAG (Seq ID No: 10) | CA (Seq ID No: 21) |
| MOKO10 | AGATCGGCAGCGTCAGATGTGTAT | AGATCGCTCTTCCGATCTACACC |
| | AAGAGACAGGAGTTATGTTATGGG | AAAAACAATTTAATAAACA (Seq |
| | AGTAAGTGGG (Seq ID No: 11) | ID No: 22) |

### Example 2: Ligation of Sequencing Adaptors with Targeted Methylation PCR Enrichment Using MoCODE

In this example, the purified PCR products treated with the restricted endonucleases in the experimental group in example 1 were ligated by virtue of the sequencing adaptors. Then, the ligation effect of the sequencing adaptors was observed by virtue of agarose gel electrophoresis.

1) Adaptor ligation (structural schematic diagrams of adaptors are shown as FIGS. 5B-C)
a) Preparation of adaptors

| | Volume (final | |
|---|---|---|
| Component | concentration) | |
| 10 × reaction buffer solution | 4 | μl |
| (100 mM Tris-HCl, pH 7.5, 10 mM EDTA) | | |
| Adaptor upper chain (200 μM) | 2 | μl |
| Adaptor lower chain (200 μM) | 2 | μl |
| Nuclease-free water | 32 | μl |
| Total volume | 40 | μl |

A resultant was incubated on a thermocycler for 2 min at 82° C.

The resultant was cooled to 25° C. at a rate of 0.1° C./3 s.

Annealing program: 82° C., 2 min; 570×{82° C., 3 s, −0.1° C./cycle}; preservation at 4° C.
b) Ligation reaction

| Component | Capacity | |
|---|---|---|
| 10 × T4 DNA ligase buffer solution (NEB) | 2 | μl |
| Purified digestion PCR product | 15 | μl |
| Forward adaptor (10 μM) | 1 | μl |
| Reverse adaptor (10 μM) | 1 | μl |
| T4 DNA ligase (NEB, 200 U/μl) | 1 | μl |
| Total volume | 20 | μl |

A reaction mixed solution was gently mixed up and down by virtue of a pipette, and transitorily centrifuged.

A resultant was incubated for 15 min at a room temperature.

2) Agarose gel electrophoresis
a) 2% agarose gel was prepared with 0.5×TBE, and a nucleic acid dye (GelSafe) was added (1 μl of dye per 10 ml of system).
b) The purified PCR product was treated with 5 μL of restricted endonuclease.
c) 150 V electrophoresis was performed for 30 minutes, and the product was photographed with a gel imaging system for observation.

3) Results of agarose gel electrophoresis

It can be clearly seen from electrophoresis results that a lengt of a product after the sequencing adaptor ligation increased by about 100 bp, indicating that adaptor ligation succeeds (FIG. 8).

4) Adaptor sequences used in this example

| Name | Adaptor upper chain (5′ > 3′) | Adaptor lower chain (5′ > 3′) |
|---|---|---|
| Forward | AATGATACGGCGACCACCGAGATCTACAC[i5] | Phos-TACACATCTGACGCT |
| adaptor | TCGTCGGCAGCGTCAGATG (Seq ID No: 23) | GCCGACGA (Seq ID No: 24) |
| Reverse | Phos-ATCGGAAGAGCACACGTCTGAACTCC | GTGACTGGAGTTCAGACG |
| adaptor | AGTCAC[i7]ATCTCGTATGCCGTCTTCTGCTT | TGTGCTCTTCC (Seq ID No: |
| | G (Seq ID No: 25) | 26) |

[i5]/[i7] represents 8 nt Illumina Index label sequence

### Example 3: Method 1 of Constructing NGS Library Using MoCODE

In this example, two different adaptors are used for constructing a library. Two MoCODE barcode sequences were generated by digesting PCR products with two restricted endonucleases.

1) Preparation of PCR template
a) Genomic DNA of Hela cells (America NEB Company) was subjected to bisulfite transformation with an EZ DNA Methylation-Gold Kit (America ZYMO Company).
b) A concentration of the transformed DNA was measured using a Qubit fluorometer.
c) A concentration of bisulfite-transformed DNA was adjusted to 50 ng/μl with water.

2) Multiplex PCR
a) PCR reaction system.

| Component | Volume | |
|---|---|---|
| Nuclease-free water | 21.5 | μl |
| 2 × KOD-Multi Epi PCR premixed solution | 25 | μl |
| Primer mixed solution (10 μM) | 1.5 | μl |
| Genomic DNA, treated with sulfite, of Hela cells | 1 μl (50 ng) | |
| KOD-Multi & Ep (TOYOBO) | 1 | μl |
| Total volume | 50 | μl |

b) PCR program
Step 1:94° C., 2 min.
Step 2: 6 cycles (98° C., 10 s; 59° C., 5 s; 68° C., 5s).
Step 3: 35 cycles (98° C., 10 s; 68° C., 10s).
Step 4: 68° C., 1 min.
Step 5: keeping at 8° C.

3) A multiplex PCR produc was purified with HiPrep PCR magnetic beads (America NEB Company)
a) The PCR product was purified with 60 μl of magnetic beads (1.2 times).
b) The purified product was eluted in 15 μl of water.
c) A concentration of the purified PCR product was measured using the Qubit fluorometer.
d) A concentration of the product was adjusted to 10 ng/μl with water.

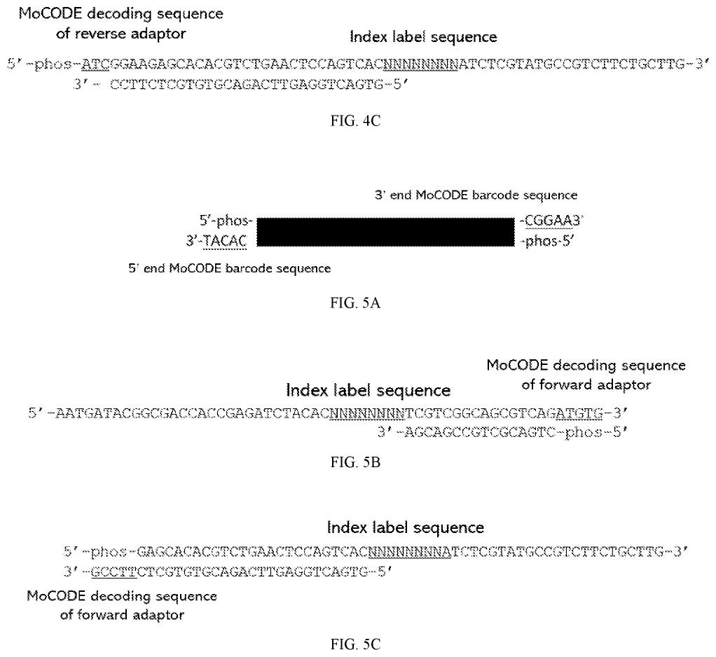

4) The purified PCR product was treated with restricted endonucleases Bbvl and Earl (the structural schematic diagram of the generated product is shown as FIG. 4A).

| Component | Volume | |
|---|---|---|
| 10 × Cutsmart buffer solution (NEB) | 2 | μl |
| BbvI (NEB, 2 U/μl) | 1 | μl |
| EarI (NEB, 20 U/μl) | 0.5 | μl |
| Purified PCR product | 5 μl 50 ng | |
| Nuclease-free water | 11.5 | μl |
| Total volume | 20 | μl |

The product was incubated on a thermocycler for 30 min at 37° C.

A resultant was incubated for 20 min at 65° C., to make the enzymes lose activity.

A reaction mixed solution was purified using HiPrep PCR magnetic beads (1.2×), and eluted in 15 μl of water.

5) Adaptor ligation (structural schematic diagrams of adaptors are shown as FIGS. 4B-C)
a) Preparation of adaptors

| | Volume (final | |
|---|---|---|
| Component | concentration) | |
| 10 × reaction buffer solution | 4 | μl |
| (100 mM Tris-HCl, pH 7.5, 10 mM EDTA) | | |
| Adaptor upper chain (200 μM) | 2 | μl |
| Adaptor lower chain (200 μM) | 2 | μl |
| Nuclease-free water | 32 | μl |
| Total volume | 40 | μl |

A resultant was incubated on a thermocycler for 2 min at 82° C.

The resultant was cooled to 25° C. at a rate of 0.1° C./3 s.

Annealing program: 82° C., 2 min; 570×{82° C., 3 s, −0.1° C./cycle}; preservation at 4° C.
b) Ligation reaction

| Component | Capacity | |
|---|---|---|
| 10 × T4 DNA ligase buffer solution (NEB) | 2 | μl |
| Purified digestion PCR product | 15 | μl |
| Forward adaptor (10 μM) | 1 | μl |
| Reverse adaptor (10 μM) | 1 | μl |
| T4 DNA ligase (NEB, 200 U/μl) | 1 | μl |
| Total volume | 20 | μl |

A reaction mixed solution was gently mixed up and down by virtue of a pipette, and transitorily centrifuged.

A resultant was incubated for 15 min at a room temperature.

A ligation mixture was purified using HiPrep PCR magnetic beads (1×), and eluted in 10 μl of water.

6) Measurement on concentration of library

1 μl of purified ligation product was taken for preparing 10-fold diluent (1:10 to 1:10,000).

A concentration of the 1:10,000 diluent was determined using a Kapa library quantification kit.

A concentration of the library was adjusted to 4 nM with water.

Sequencing was performed on the Illumina sequencing platform.

7) Sequencing results

An original .fastq file for Illumina double-end sequencing was assembled into a complete tested segment by virtue of PEAR software. The sequencing results after each assembly were compared with the target segment sequence. A sequence, meeting an expected read, generated by the correct paired primers is identified as on-target, and an on-target rate is a proportion of a number of on-target sequences in a total number of reads.

Total number of reads: 554265; on-target rate: 97.0%.

8) PCR primer sequences used in this example

As shown in the following, universal specific molecular barcode generating sequences of the forward primer and the reverse primer, and the sequences of the Moko1-10 forward primer and reverse primer are the same as those in example 1. The Moko11-23 forward primer includes sequences shown as Seq ID Nos: 27, 29, 31, 33, 35, 37, 39, 41, 43, 45, 47, 49, 51; and the Moko11-23 reverse primer includes sequences shown as Seq ID Nos: 28, 30, 32, 34, 36, 38, 40, 42, 44, 46, 48, 50, 52.

| Name | Forward primer (5′ > 3′) | Reverse primer (5′ > 3′) |
|---|---|---|
| Universal specific | AGATCGGCAGCGTCAGATGT | AGATCGCTCTTCCGATCT (Seq |
| molecular barcode | GTATAAGAGACAG (Seq ID | ID No: 12) |
| generating | No: 1) | |
| sequence (5′ > 3′) | | |
| Moko1 | AGATCGGCAGCGTCAGATGT | AGATCGCTCTTCCGATCTATAT |
| | GTATAAGAGACAGGAGTAGT | ATATCAAACACTRGACTTAAA |
| | TGGGATTATAGGTGT (Seq ID | AT (Seq ID No: 13) |
| | No: 2) | |
| Moko2 | AGATCGGCAGCGTCAGATGT | AGATCGCTCTTCCGATCTCCTT |
| | GTATAAGAGACAGTTAGAAA | AAAACAAACTTATCTTCTCC |
| | TTTAGTTGTAGAGGGGG (Seq | (Seq ID No: 14) |
| | ID No: 3) | |
| Moko3 | AGATCGGCAGCGTCAGATGT | AGATCGCTCTTCCGATCTCAC |
| | GTATAAGAGACAGGAGGTTA | CTTAACAAATAAAATAATAATT |
| | GGGTTTTAGATTGGGA (Seq | CAC (Seq ID No: 15) |
| | ID No: 4) | |
| Moko4 | AGATCGGCAGCGTCAGATGT | AGATCGCTCTTCCGATCTTATA |
| | GTATAAGAGACAGGTAAYGA | CTAACTCCCTTCAACCATTA |
| | ATTGGTAGAGTTTTA (Seq ID | (Seq ID No: 16) |
| | No: 5) | |
| Moko5 | AGATCGGCAGCGTCAGATGT | AGATCGCTCTTCCGATCTCTAC |
| | GTATAAGAGACAGTGAGGGT | CCACACCTACCAAACCTAA |
| | AAGAATTATTTAGAGGT (Seq | (Seq ID No: 17) |
| | ID No: 6) | |
| Moko6 | AGATCGGCAGCGTCAGATGT | AGATCGCTCTTCCGATCTATCA |
| | GTATAAGAGACAGAGGGTTA | AAAATAATTCTAAAAATATACA |
| | AAGAAGAGAATGATTTAT | (Seq ID No: 18) |
| | (Seq ID No: 7) | |
| Moko7 | AGATCGGCAGCGTCAGATGT | AGATCGCTCTTCCGATCTACCA |
| | GTATAAGAGACAGGAGGGTT | ACTTCTATATAACTAATAAATA |
| | GAATATTAAAAATAGTAGGG | CACA (Seq ID No: 19) |
| | T (Seq ID No: 8) | |
| Moko8 | AGATCGGCAGCGTCAGATGT | AGATCGCTCTTCCGATCTAAAA |
| | GTATAAGAGACAGGGATAAT | TTCACTTCTAAATTTAAACCA |
| | TATAAGAATTGTAAAGGAGG | (Seq ID No: 20) |
| | AT (Seq ID No: 9) | |
| Moko9 | AGATCGGCAGCGTCAGATGT | AGATCGCTCTTCCGATCTAAAA |
| | GTATAAGAGACAGGGTAGTT | TAATCTTCATCAAATTAATAAA |
| | GGAAATGGTAAATTTGAG | AACA (Seq ID No: 21) |
| | (Seq ID No: 10) | |
| Moko10 | AGATCGGCAGCGTCAGATGT | AGATCGCTCTTCCGATCTACAC |
| | GTATAAGAGACAGGAGTTAT | CAAAAACAATTTAATAAACA |
| | GTTATGGGAGTAAGTGGG | (Seq ID No: 22) |
| | (Seq ID No: 11) | |
| Moko11 | AGATCGGCAGCGTCAGATGT | AGATCGCTCTTCCGATCTTTTT |
| | GTATAAGAGACAGTTAGGGT | ACCAAAACTAATACTAACAAC |
| | TTTAGATTGGGAGG (Seq ID | T (Seq ID No: 28) |
| | No: 27) | |
| Moko12 | AGATCGGCAGCGTCAGATGT | AGATCGCTCTTCCGATCTAATC |
| | GTATAAGAGACAGGTTAGGG | AATCTCTCTAAACCAAAAA |
| | AAGTTGATGTTAGGAAAT | (Seq ID No: 30) |
| | (Seq ID No: 29) | |
| Moko13 | AGATCGGCAGCGTCAGATGT | AGATCGCTCTTCCGATCTAATA |
| | GTATAAGAGACAGTAGTTATA | CAAATCAATAAATTTACATACA |
| | TGGAAAGTTGAGATAGAAGG | AAA (Seq ID No: 32) |
| | A (Seq ID No: 31) | |
| Moko14 | AGATCGGCAGCGTCAGATGT | AGATCGCTCTTCCGATCTACAT |
| | GTATAAGAGACAGAAGAATA | AATAAAACCCTATCTCTACTAA |
| | ATTTAATAGGATTGGAAGGA | AAA (Seq ID No: 34) |
| | AT (Seq ID No: 33) | |
| Moko15 | AGATCGGCAGCGTCAGATGT | AGATCGCTCTTCCGATCTTAAA |
| | GTATAAGAGACAGTATAGGT | TCCTTAAATAAACTACATAAAA |
| | GATTTTAGGGGTGAGA (Seq | ATTTTCC (Seq ID No: 36) |
| | ID No: 35) | |
| Moko16 | AGATCGGCAGCGTCAGATGT | AGATCGCTCTTCCGATCTACCA |
| | GTATAAGAGACAGGAGGTAG | ACTATACCTCTACATCAAAA |
| | TAATAGGGAAAATAGTTATTG | (Seq ID No: 38) |
| | G (Seq ID No: 37) | |
| Moko17 | AGATCGGCAGCGTCAGATGT | AGATCGCTCTTCCGATCTAAA |
| | GTATAAGAGACAGAAGGGG | ACCTATATCTCTAATAAAAACT |
| | GAATTTTAGTTTTAGGAA | CAATA (Seq ID No: 40) |
| | (Seq ID No: 39) | |
| Moko18 | AGATCGGCAGCGTCAGATGT | AGATCGCTCTTCCGATCTAAA |
| | GTATAAGAGACAGTTTGTTTT | ACCCCAACATTCAATTAAAAA |
| | AGGAAAGAGGTGG (Seq ID | (Seq ID No: 42) |
| | No: 41) | |
| Moko19 | AGATCGGCAGCGTCAGATGT | AGATCGCTCTTCCGATCTAAC |
| | GTATAAGAGACAGAATAATG | ACCATCTCAACTCACTACAAA |
| | TAATAAGAATAAAAGGTAAG | CT (Seq ID No: 44) |
| | GTT (Seq ID No: 43) | |
| Moko20 | AGATCGGCAGCGTCAGATGT | AGATCGCTCTTCCGATCTCCCC |
| | GTATAAGAGACAGGAGTATT | AACCTCTAATATATATACCCAA |
| | GGGGATTTAGGGG (Seq ID | (Seq ID No: 46) |
| | No: 45) | |
| Moko21 | AGATCGGCAGCGTCAGATGT | AGATCGCTCTTCCGATCTAACC |
| | GTATAAGAGACAGGGATAAA | ACAAATAAAATATAAATACTCA |
| | GTAAAGGAGATATTGTATGG | TAAA (Seq ID No: 48) |
| | AA (Seq ID No: 47) | |
| Moko22 | AGATCGGCAGCGTCAGATGT | AGATCGCTCTTCCGATCTAACC |
| | GTATAAGAGACAGGGAGGA | TCTTTATTTACAAACCTAAAC |
| | AAGAGAATATTTGATATTTG | (Seq ID No: 50) |
| | (Seq ID No: 49) | |
| Moko23 | AGATCGGCAGCGTCAGATGT | AGATCGCTCTTCCGATCTCACT |
| | GTATAAGAGACAGTATTTTAA | TCCTAAAACRGAAAAATTCTA |
| | TCTCCTCACCAACAAAAA | (Seq ID No: 52) |
| | (Seq ID No: 51) | |

Underlined is a specific target gene sequence

9) Adaptor sequences used in this example

As shown in following, the used adaptor sequences are the same as that in example 2 (eq ID Nos: 23-26).

| Name | Adaptor upper chain (5′ > 3′) | Adaptor lower chain (5′ > 3′) |
|---|---|---|
| Forward adaptor | AATGATACGGCGACCACCGAGATCT | Phos-TACACATCTGACGC |
| | ACAC[i5]TCGTCGGCAGCGTCAGATG | TGCCGACGA (Seq ID No: |
| | (Seq ID No: 23) | 24) |
| Reverse adaptor | Phos-ATCGGAAGAGCACACGTCTGA | GTGACTGGAGTTCAGAC |
| | ACTCCAGTCAC[i7]ATCTCGTATGCC | GTGTGCTCTTCC (Seq ID |
| | GTCTTCTGCTTG (Seq ID No: 25) | No: 26) |

[i5]/[i7] represents 8 nt Illumina Index label sequence

10) MoCODE barcode sequences and MoCODE barcode decoding sequences used in this example

| | MoCODE barcode | MoCODE barcode decoding |
|---|---|---|
| | sequence (5′ > 3′) | sequence (5′ > 3′) |
| Forward | TGTA (Seq ID No: 53) | TACA (Seq ID No: 54) |
| adaptor | | |
| Reverse | GAT (Seq ID No: 55) | ATC (Seq ID No: 56) |
| adaptor | | |

### Example 4: Method 2 of Constructing NGS Library Using MoCODE

In this example, two different adaptors are used for constructing a library. Two MoCODE barcode sequences were generated by digesting the PCR products with one endonuclease.

1) Preparation of PCR template
a) 1-1.5 ml of to-be-tested Thin-Cytologic Test/Liquid-based cytologic test (TCT/LCT) cell preservation solution was centrifuged, and a supernatant was removed; then 200 ml of PBS was added for resuspension; and DNA was extracted using a DNeasy Blood & Tissue Kit (Germany QIAGEN Company).
b) A concentration of the obtained DNA was measured using a Qubit fluorometer.
c) The obtained DNA was subjected to bisulfite transformation with an EZ DNA Methylation-Gold Kit (America ZYMO Company).
e) A concentration of the transformed DNA was measured using a Qubit fluorometer.
d) A concentration of bisulfite-transformed DNA was adjusted to 10 ng/μl with water.

2) Multiplex PCR
a) PCR reaction system

| Component | Volume | |
|---|---|---|
| Nuclease-free water | 17.5 | μl |
| 2 × KOD-Multi Epi PCR premixed solution (TOYOBO) | 25 | μl |
| Primer mixed solution (10 μM) | 1.5 | μl |
| Genomic DNA treated with sulfite | 5 μl (50 ng) | |
| KOD-Multi & Ep (TOYOBO) | 1 | μl |
| Total volume | 50 | μl |

b) PCR program;
Step 1:94° C., 2 min.
Step 2: 6 cycles (98° C., 10 s; 59° C., 5 s; 68° C., 5s).
Step 3: 35 cycles (98° C., 10 s; 64° C., 5 s; 68° C., 5s).
Step 4: 68° C., 1 min.
Step 5: keeping at 8° C.

3) Purification of multiplex PCR product with AMPure XP magnetic beads (America Beckman Coulter Company)
a) The PCR product was purified with 75 μl of magnetic beads (1.5 times).
b) The purified product was eluted in 15 μl of water.
c) A concentration of the purified PCR product was measured using the Qubit fluorometer.
d) A concentration of the product was adjusted to 20 ng/μl with water.

4) The purified PCR product was treated with Endonuclease V (America NEB Company) (the structural schematic diagram of the generated product is shown as FIG. 5A).

| Component | Volume | |
|---|---|---|
| 10 × buffer solution 4 (NEB) | 2 | μl |
| Endonuclease V (NEB, 10 U/μl) | 1 | μl |
| Purified PCR product | 5 μl (100 ng) | |
| Nuclease-free water | 12 | μl |
| Total volume | 20 | μl |

The product was incubated on a thermocycler for 30 min at 37° C.

A resultant was incubated for 20 min at 65° C., to make the enzymes lose activity.

A reaction mixed solution was purified using AMPure XP magnetic beads (1.5×), and eluted in 13 μl of water.

5) Adaptor ligation
a) Preparation of adaptor (structural schematic diagrams of adaptors are shown as FIGS. 5B-C)

| | Volume (final | |
|---|---|---|
| Component | concentration) | |
| 10 × reaction buffer solution | 4 | μl |
| (100 mM Tris-HCl, pH 7.5, 10 mM EDTA) | | |
| Adaptor upper chain (200 μM) | 2 | μl |
| Adaptor lower chain (200 μM) | 2 | μl |
| Nuclease-free water | 32 | μl |
| Total volume | 40 | μl |

A resultant was incubated on a thermocycler for 2 min at 82° C.

The resultant was cooled to 25° C. at a rate of 0.1° C./3 s.

Annealing program: 82° C., 2 min; 570×{82° C., 3 s, −0.1° C./cycle}; preservation at 4° C.
b) Ligation reaction

| Component | Capacity | |
|---|---|---|
| 10 × T4 DNA ligase buffer solution (NEB) | 2 | μl |
| Purified digestion PCR product | 13 | μl |
| Forward adaptor (10 μM) | 2 | μl |
| Reverse adaptor (10 μM) | 2 | μl |
| T4 DNA ligase (NEB, 200 U/μl) | 1 | μl |
| Total volume | 20 | μl |

A reaction mixed solution was gently mixed up and down by virtue of a pipette, and transitorily centrifuged.

A resultant was incubated for 15 min at a room temperature.

A ligation mixture was purified using the AMPure XP magnetic beads (1.2×), and eluted in 10 μl of water.

6) Measurement on concentration of library
a) 1 μl of purified ligation product was taken for preparing 10-fold diluent (1:10 to 1:10,000).
b) A concentration of the 1:10,000 diluent was determined using a Kapa library quantification kit.
c) A concentration of the library was adjusted to 4 nM with water.
d) Sequencing was performed on the Illumina sequencing platform.

7) Sequencing results

An original .fastq file for Illumina double-end sequencing was assembled into a complete tested segment by virtue of PEAR software. The sequencing results after each assembly were compared with the target segment sequence. A sequence, meeting an expected read, generated by the correct paired primers is identified as on-target, and an on-target rate is a proportion of a number of on-target sequences in a total number of reads.

| | Sample 1 | Sample 2 |
|---|---|---|
| Total number of reads | 1225399 | 1143004 |
| On-target rate | 98.0% | 98.2% |

8) PCR primer sequences used in this example

As shown in the following, they are Seq ID Nos: 57-104 from left to right and from top to bottom.

| Name | Forward primer (5′ > 3′) | Reverse primer (5′ > 3′) |
|---|---|---|
| Universal specific | ATGTITATAAGAGACAG (Seq ID | TTCCIATC (Seq ID No: 58) |
| molecular barcode | No: 57) | |
| generating | | |
| sequence (5′ > 3′) | | |
| Mokola | ATGTITATAAGAGACAGGAGTAG | TTCCIATCATATATATCAAAC |
| | TTGGGATTATAGGTGT (Seq ID | ACTRGACTTAAAAT (Seq ID |
| | No: 59) | No: 60) |
| Moko2a | ATGTITATAAGAGACAGTTAGAA | TTCCIATCCCTTAAAACAAA |
| | ATTTAGTTGTAGAGGGGG (Seq | CTTATCTTCTCC (Seq ID No: |
| | ID No: 61) | 62) |
| Moko3a | ATGTITATAAGAGACAGGAGGTT | TTCCIATCCACCTTAACAAA |
| | AGGGTTTTAGATTGGGA (Seq ID | TAAAATAATAATTCAC (Seq |
| | No: 63) | ID No: 64) |
| Moko4a | ATGTITATAAGAGACAGGTAAYG | TTCCIATCTATACTAACTCCC |
| | AATTGGTAGAGTTTTA (Seq ID | TTCAACCATTA (Seq ID No: |
| | No: 65) | 66) |
| Moko5a | ATGTITATAAGAGACAGTGAGG | TTCCIATCCTACCCACACCT |
| | GTAAGAATTATTTAGAGGT (Seq | ACCAAACCTAA (Seq ID No: |
| | ID No: 67) | 68) |
| Moko6a | ATGTITATAAGAGACAGAGGGTT | TTCCIATCATCAAAAATAAT |
| | AAAGAAGAGAATGATTTAT (Seq | TCTAAAAATATACA (Seq ID |
| | ID No: 69) | No: 70) |
| Moko7a | ATGTITATAAGAGACAGGAGGG | TTCCIATCACCAACTTCTATA |
| | TTGAATATTAAAAATAGTAGGGT | TAACTAATAAATACACA |
| | Seq ID No: 71) | (Seq ID No: 72) |
| Moko8a | ATGTITATAAGAGACAGGGATAA | TTCCIATCAAAATTCACTTC |
| | TTATAAGAATTGTAAAGGAGGA | TAAATTTAAACCA (Seq ID |
| | T (Seq ID No: 73) | No: 74) |
| Moko9a | ATGTITATAAGAGACAGGGTAGT | TTCCIATCAAAATAATCTTC |
| | TGGAAATGGTAAATTTGAG (Seq | ATCAAATTAATAAAAACA |
| | ID No: 75) | (Seq ID No: 76) |
| Moko10a | ATGTITATAAGAGACAGGAGTTA | TTCCIATCACACCAAAAAC |
| | TGTTATGGGAGTAAGTGGG (Seq | AATTTAATAAACA (Seq ID |
| | ID No: 77) | No: 78) |
| Moko11a | ATGTITATAAGAGACAGTTAGGG | TTCCIATCTTTTACCAAAAC |
| | TTTTAGATTGGGAGG (Seq ID No: | TAATACTAACAACT (Seq ID |
| | 79) | No: 80) |
| Moko12a | ATGTITATAAGAGACAGGTTAGG | TTCCIATCAATCAATCTCTCT |
| | GAAGTTGATGTTAGGAAAT Seq | AAACCAAAAA (Seq ID No: |
| | ID No: 81) | 82) |
| Moko13a | ATGTITATAAGAGACAGTAGTTA | TTCCIATCAATACAAATCAA |
| | TATGGAAAGTTGAGATAGAAGG | TAAATTTACATACAAAA |
| | A (Seq ID No: 83) | (Seq ID No: 84) |
| Moko14a | ATGTITATAAGAGACAGAAGAAT | TTCCIATCACATAATAAAAC |
| | AATTTAATAGGATTGGAAGGAA | CCTATCTCTACTAAAAA (Seq |
| | T (Seq ID No: 85) | ID No: 86) |
| Moko15a | ATGTITATAAGAGACAGTATAGG | AGATCGCTCTTCCGATCTTA |
| | TGATTTTAGGGGTGAGA (Seq ID | AATCCTTAAATAAACTACAT |
| | No: 87) | AAAAA (Seq ID No: 88) |
| Moko16a | ATGTITATAAGAGACAGGAGGTA | TTCCIATCACCAACTATACC |
| | GTAATAGGGAAAATAGTTATTG | TCTACATCAAAA (Seq ID No: |
| | G (Seq ID No: 89) | 90) |
| Moko17a | ATGTITATAAGAGACAGAAGGG | TTCCIATCAAAACCTATATC |
| | GGAATTTTAGTTTTAGGAA (Seq | TCTAATAAAAACTCAATA |
| | ID No: 91) | (Seq ID No: 92) |
| Moko18a | ATGTITATAAGAGACAGTTTGTT | TTCCIATCAAAACCCCAACA |
| | TTAGGAAAGAGGTGG (Seq ID | TTCAATTAAAAA (Seq ID No: |
| | No: 93) | 94) |
| Moko19a | ATGTITATAAGAGACAGAATAAT | TTCCIATCAACACCATCTCA |
| | GTAATAAGAATAAAAGGTAAGG | ACTCACTACAAACT (Seq ID |
| | TT (Seq ID No: 95) | No: 96) |
| Moko20a | ATGTITATAAGAGACAGGAGTAT | TTCCIATCCCCCAACCTCTA |
| | TGGGGATTTAGGGG (Seq ID No: | ATATATATACCCAA (Seq ID |
| | 97) | No: 98) |
| Moko21a | ATGTITATAAGAGACAGGGATAA | TTCCIATCAACCACAAATAA |
| | AGTAAAGGAGATATTGTATGGA | AATATAAATACTCATAAA |
| | A (Seq ID No: 99) | (Seq ID No: 100) |
| Moko22a | ATGTITATAAGAGACAGGGAGG | TTCCIATCAACCTCTTTATTT |
| | AAAGAGAATATTTGATATTTG | ACAAACCTAAAC (Seq ID |
| | (Seq ID No: 101) | No: 102) |
| Moko23a | ATGTITATAAGAGACAGTATTTT | TTCCIATCCACTTCCTAAAA |
| | AATCTCCTCACCAACAAAAA | CRGAAAAATTCTA (Seq ID |
| | (Seq ID No: 103) | No: 104) |

I:dITP

A sequence fragment as underlined is a specific target gene sequence

9) Adaptor sequences used in this example

As shown in following, they are Seq ID Nos: 105-108 sequentially.

| Name | Adaptor upper chain (5′ > 3′) | Adaptor lower chain (5′ > 3′) |
|---|---|---|
| Forward | AATGATACGGCGACCACCGAGAT | phos- |
| adaptor | CTACAC[i5]TCGTCGGCAGCGTCA | CTGACGCTGCCGACGA |
| | GATGTG (Seq ID No: 105) | (Seq ID No: 106) |
| Reverse | Phos-GAGCACACGTCTGAACTCC | GTGACTGGAGTTCAGACG |
| adaptor | AGTCAC[i7]ATCTCGTATGCCGTCT | TGTGCTCTTCCG (Seq ID |
| | TCTGCTTG (Seq ID No: 107) | No: 108) |

[i5]/[i7] represents 8 nt Illumina Index label sequence

10) MoCODE barcode sequences and MoCODE barcode decoding sequences used in this example

As shown in following, they are Seq ID Nos: 109-112 sequentially.

| | MoCODE barcode | MoCODE barcode decoding |
|---|---|---|
| | sequence (5′ > 3′) | sequence (5′ > 3′) |
| Forward adaptor | CACAT (Seq ID No: 109) | ATGTG (Seq ID No: 110) |
| Reverse adaptor | CGGAA (Seq ID No: 111) | TTCCG (Seq ID No: 112) |

What is claimed is:

1. A method for constructing a multiplex PCR library for high-throughput targeted sequencing, characterized in that, by adding polybasic MoCODE barcodes to a specific amplification product, and using the MoCODE barcodes to efficiently ligating the amplification product to sequencing adaptors comprising MoCODE barcode decoding sequences, a library is constructed; the MoCODE barcodes refer to overhanging single-stranded nucleotide sequences constituting two sticky ends of an obtained PCR product after the multiplex PCR product is digested with a specific endonuclease; and the MoCODE barcode decoding sequences are nucleotide sequences complementary to the MoCODE barcodes.

2. The method of claim 1, wherein a generation mode of the MoCODE barcodes comprises: one or more of a modified nucleotide, a nicking enzyme, an endonuclease, chemical modification, base photodegradation and the like; preferably, the modified nucleotide comprises one or more of dUTP, dITP and an RNA base.

3. The method of claim 1, wherein the MoCODE barcodes may be the same or different within molecules.

4. The method of claim 1, wherein the MoCODE barcodes are non-random specific barcodes.

5. The method of claim 1, wherein the MoCODE barcode has a length of 2-20 nt, and preferably, the MoCODE barcode decoding sequence is complementary to a MoCODE barcode sequence, having a length of 2-20 nt.

6. The method of claim 1, wherein the sequencing adaptor may be artificially designed and synthesized or matched with an own fragment sequence of a target segment; preferably, each sequencing adaptor may be a single adaptor and a bidirectional adaptor; preferably, enrichment in each specific segment may be decoded by virtue of the single adaptor, the bidirectional adaptor or automatic cyclization.

7. A primer for multiplex PCR for high-throughput targeted sequencing, characterized in that the primer comprises a MoCODE barcode generating sequence; preferably, the primer comprises the sequence selected from sequences shown as Seq ID Nos: 1-22, 27-52, 53, 55, 57-104, 109 and 111.

8. A sequencing adaptor for multiplex PCR for high-throughput targeted sequencing, characterized in that the sequencing adaptor comprises a MoCODE barcode decoding sequence; preferably, the sequencing adaptor further comprises one or more of a sequencing adaptor of a sequencing platform and an index label; preferably, the sequencing adaptor comprises a universal sequence for high-throughput sequencing, an index label and a MoCODE barcode decoding sequence; preferably, the sequencing adaptor comprises the sequence selected from sequences shown as Seq ID Nos: 23-26, 54, 56, 105-108, 110 and 112.

9. A method for constructing a multiplex PCR library for high-throughput targeted sequencing, characterized in that the method comprises the following steps:
1) extracting DNA from a to-be-tested specimen;
2) performing a multiplex PCR reaction, each primer, participating to the multiplex PCR reaction, comprising a specific MoCODE barcode generating sequence; preferably, the primer further comprising a gene-specific sequence;
3) purifying a PCR product obtained in step 2) with magnetic beads;
4) making the PCR product purified in step 3) generate a 5′ sticky end and a 3′ sticky end, and generating MoCODE barcodes at the 5′ sticky end and the 3′ sticky end respectively;
5) purifying the PCR product comprising the MoCODE barcodes in step 4) with the magnetic beads;
6) ligating the PCR product, comprising the MoCODE barcodes, purified in step 5) to the sequencing adaptors, the sequencing adaptor comprising MoCODE barcode decoding sequences complementary to the MoCODE barcodes;
7) purifying a ligation product obtained in step 6) with the magnetic beads, and completing construction of the multiplex PCR library for high-throughput targeted sequencing.

10. The method of claim 9, wherein in step 4), a generation mode of the MoCODE barcode comprises: one or more of a modified nucleotide, a nicking enzyme, an endonuclease, chemical modification, base photodegradation and the like; preferably, the modified nucleotide comprises one or more of dUTP, dITP and an RNA base, and more preferably, the generation mode of the MoCODE barcodes is to use a specific endonuclease for digestion;
preferably, in step 4), one MoCODE barcode is generated at each of the 5′ sticky end and the 3′ sticky end, wherein the MoCODE barcodes at the 5′ sticky end and the 3′ sticky end may be same or different;
preferably, in step 6), each sequencing adaptor may be a single adaptor, a bidirectional adaptor or a cyclization adaptor.

11. The method of claim 2, wherein the MoCODE barcodes may be the same or different within molecules.

12. The method of claim 2, wherein the MoCODE barcodes are non-random specific barcodes.

13. The method of claim 3, wherein the MoCODE barcodes are non-random specific barcodes.

14. The method of claim 2, wherein the MoCODE barcode has a length of 2-20 nt, and preferably, the MoCODE barcode decoding sequence is complementary to a MoCODE barcode sequence, having a length of 2-20 nt.

15. The method of claim 3, wherein the MoCODE barcode has a length of 2-20 nt, and preferably, the MoCODE barcode decoding sequence is complementary to a MoCODE barcode sequence, having a length of 2-20 nt.

16. The method of claim 4, wherein the MoCODE barcode has a length of 2-20 nt, and preferably, the MoCODE barcode decoding sequence is complementary to a MoCODE barcode sequence, having a length of 2-20 nt.

17. The method of claim 2, wherein the sequencing adaptor may be artificially designed and synthesized or matched with an own fragment sequence of a target segment; preferably, each sequencing adaptor may be a single adaptor and a bidirectional adaptor; preferably, enrichment in each specific segment may be decoded by virtue of the single adaptor, the bidirectional adaptor or automatic cyclization.

18. The method of claim 3, wherein the sequencing adaptor may be artificially designed and synthesized or matched with an own fragment sequence of a target segment; preferably, each sequencing adaptor may be a single adaptor and a bidirectional adaptor; preferably, enrichment in each specific segment may be decoded by virtue of the single adaptor, the bidirectional adaptor or automatic cyclization.

19. The method of claim 4, wherein the sequencing adaptor may be artificially designed and synthesized or matched with an own fragment sequence of a target segment; preferably, each sequencing adaptor may be a single adaptor and a bidirectional adaptor; preferably, enrichment in each specific segment may be decoded by virtue of the single adaptor, the bidirectional adaptor or automatic cyclization.

20. The method of claim 5, wherein the sequencing adaptor may be artificially designed and synthesized or matched with an own fragment sequence of a target segment; preferably, each sequencing adaptor may be a single adaptor and a bidirectional adaptor; preferably, enrichment in each specific segment may be decoded by virtue of the single adaptor, the bidirectional adaptor or automatic cyclization.
